SEQUENCE LISTING (1 sequence)

SEQ ID NO: 1 - protein, 31 aa, synthetic construct
KVGEKLSEGDLLAEIETDKATIGFEVQEEGY

(19) United States
(12) Patent Application Publication
(10) Pub. No.: US 2026/0306598 A1
(43) Pub. Date: Oct. 8, 2026

(54) PREPARATION OF TOLERIZING NANOPARTICLES FOR THE TREATMENT OF PRIMARY BILIARY CHOLANGITIS

(71) Applicant: COUR PHARMACEUTICALS DEVELOPMENT COMPANY INC., Evanston, IL (US)

(72) Inventors: Adam Elhofy, Chicago, IL (US); Tushar Murthy, Northbrook, IL (US); Michael Boyne, Skokie, IL (US)

(21) Appl. No.: 19/485,809

(22) PCT Filed: May 23, 2024
(86) PCT No.: PCT/US2024/030775

§ 371 Date: Nov. 19, 2025

Related U.S. Application Data
(60) Provisional application No. 63/503,771, filed on May 23, 2023.

(51) Int. Cl. A61K 39/00; A61K 9/51

(52) U.S. Cl. CPC A61K 39/0008; A61K 9/5146; A61K 9/5153; A61K 9/5192; A61K 2039/55555; A61K 2039/577

(57) ABSTRACT

The present disclosure relates to a process for the preparation of tolerizing immune modifying nanoparticles encapsulating antigens associated with primary biliary cholangitis (PBC), compositions comprising the particles and use thereof for the treatment of PBC.

### CROSS-REFERENCE TO RELATED APPLICATIONS

This application claims the priority benefit of U.S. Provisional Patent Application No. 63/503,771, filed May 23, 2023, herein incorporated by reference in its entirety.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: Filename: 58505_SeqListing.xml; Size: 1,972 bytes; Created: May 20, 2024.

### FIELD OF THE DISCLOSURE

The present disclosure relates to the process for the preparation of tolerizing immune modifying nanoparticles encapsulating primary biliary cholangitis (PBC) associated antigens.

### BACKGROUND

Primary biliary cholangitis (PBC, previously referred to as primary biliary cirrhosis) is a prototypical autoimmune liver disease characterized by destructive lymphocytic cholangitis and specific anti-mitochondrial autoantibodies (AMAs) targeted primarily at the E2 component of the mitochondrial pyruvate dehydrogenase complex (PDC-E2).¹ The prevalence of PBC in the United States is higher in women than in men and is estimated at approximately 580 per million women.² There is a significant unmet medical need with this rare disease and an opportunity where TIMP-PBC will positively impact subjects suffering from this disease.

Tolerizing immune modifying particles (TIMPs), comprising one or more antigens, have been previously described for the treatment of immune-mediated disorders (e.g., autoimmune diseases and allergies) via induction of antigen-specific immune tolerance (WO2013192532 and WO2015023796 incorporated herein by reference).

### SUMMARY

Encapsulation of one or more primary biliary cholangitis (PBC)-associated antigens within the TIMP core (TIMP-PBC) is an advantage as it ensures delivery of encapsulated proteins to APCs safely and effectively without inducing immune activation.

The process for manufacturing of TIMP-PBC involves numerous steps each of which influences the physiochemical properties of resulting compositions essential for safe and therapeutic administration. Importantly, the process must be developed to ensure efficient encapsulation of PBC antigens within the particle core.

In various embodiments, TIMP-PBC made by the method herein encapsulates one or more PBC associated antigens or antigenic epitopes thereof. In various embodiments, the primary biliary cholangitis (PBC)-associated antigens comprise intracellular proteins, extracellular proteins, mitochondrial proteins, and/or nuclear proteins. In various embodiments, the PBC antigens are selected from the group consisting of pyruvate dehydrogenase complex E2 subunit (PDC-E2), gp210, nucleoporin 62, Sp100, PML, CENP A, CENP B, CENP C, dsDNA, histone, branched-chain 2-oxo-acid dehydrogenase complex (BCOADC) 2-oxo-acid dehydrogenase complex, lipoamide acyltransferase component of branched-chain alpha-keto acid dehydrogenase complex (BCOADC-E2), dihydrolipoyllysine-residue succinyltransferase component of 2-oxoglutarate dehydrogenase complex (OGDC-E2), sulfite oxidase, outer mitochondrial membrane, glycogen phosphorylase, sarcosine dehydrogenase, smooth muscle protein, soluble liver antigen, liver/kidney microsome, centromere protein, tubulin, actin, vimentin, desmin, cytokeratin, F-actin, UDP glucuronosyltransferase Family 1 Member A Complex (UGTA1), formiminotransferase cyclodeaminase, asialoglycoprotein receptor (ASGPR), cardiolipin, h-Lamp-2, proteinase 3, CYP 2C9, CYP 2A6, and CYP P450 2D6. In some embodiments, the PBC-associated antigens are selected from the group comprising bacterial epitopes, viral epitopes, or xenobiotics. In some embodiments the bacterial epitopes, viral epitopes, or xenobiotics comprise Novosphingobium aromaticivorans proteins Novo 1, Novo 2, Novo 3, Novo 4, Lactobacillus delbrueckii beta-galactosidase, 2-octynamide, 2-nonynamide, Escherichia coli PDC-E2, Escherichia coli ATP dependent ClpX, Escherichia coli periplasmic maltose-binding protein, Escherichia coli ATP dependent helicase Hrp, Escherichia coli fatty acid oxidation complex alpha, Escherichia coli ppGpp synthetase II, Escherichia coli nitrate reductase 2, Helicobacter pylori urease beta subunit, Pseudomonas aeruginosa diaminopimelate decarboxylase, human cytomegalovirus capsid assembly protein UL47, Haemophilus influenzae t-RNA (uracil-5-)-methyltransferase. In various embodiments, the TIMP-PBC particles encapsulate one or more polynucleotides encoding PBC associated antigens. In some embodiments, the polynucleotides comprise DNA, RNA, messenger RNA (mRNA), or circular RNA.

In various embodiments, TIMP-PBC encapsulates pyruvate dehydrogenase complex E2 subunit (PDC-E2) peptides comprising antigenic epitopes. In various embodiments, TIMP-PBC encapsulates a PDC-E2 antigenic epitope comprising amino acids 155-185 (PDC-E2₁₅₅₋₁₈₅), having amino acid sequence KVGEKLSEGDLLAEIETDKATIGFEVQEEGY) (SEQ ID NO: 1).

The present disclosure provides a process for manufacturing a composition comprising negatively charged particles encapsulating one or more PBC associated antigens (TIMP-PBC). The process is directed to manufacturing particles developed for safe and therapeutic administration of TIMP-PBC for the treatment of PBC. In various embodiments, the method comprises: (a) generating primary emulsion particles by mixing one or more PBC associated antigens with an oil phase including a polymer; (b) mixing the primary emulsion particles with one or more surfactants and/or stabilizers; (c) homogenizing the mixture of (b) to form secondary emulsion particles; (d) hardening the secondary emulsion particles.

In various embodiments, the method comprises: (a) generating an aqueous solution of one or more PBC associated antigens; (b) generating a primary emulsion by mixing the aqueous solution of step (a) with an oil phase including a polymer; (c) mixing the primary emulsion of step (b) with a mixture including one or more surfactants and/or stabilizers to form a secondary emulsion; (d) hardening the secondary emulsion by evaporation resulting in hardened polymeric nanoparticles encapsulating PBC associated antigens within their cores.

In various embodiments, the method further comprises the step (e) filtering, washing, and concentrating the nanoparticles. In various embodiments, the method further comprises (e) filtering, washing, and concentrating the nanoparticles and (f) freeze drying the nanoparticles. In various embodiments, the primary emulsion of step (b) is a water-in-oil emulsion. In various embodiments, the secondary emulsion of step (c) is an oil-in-water emulsion.

In various embodiments, the method comprises: (a) generating an aqueous solution of one or more PBC associated antigens; (b) generating a primary emulsion by mixing the aqueous solution of step (a) with an oil phase including a polymer; (c) mixing the primary emulsion of step (b) with a mixture including one or more surfactants and/or stabilizers to form a secondary emulsion; (d) hardening the secondary emulsion by evaporation to remove the solvent resulting in hardened polymeric nanoparticles encapsulating PBC associated antigens within their cores; (c) filtering, washing, and concentrating the nanoparticles; and (f) freeze drying the nanoparticles.

In various embodiments, the one or more PBC associated antigens in step (a) are dissolved in an aqueous solution comprising a solvent. In various embodiments, the one or more PBC associated antigens in step (a) are dissolved in an aqueous solution comprising the same solvent. In various embodiments, one or more PBC associated antigens in step (a) are dissolved in an aqueous solution comprising different solvents. In various embodiments, one or more PBC associated antigens in step (a) are dissolved in an aqueous solution comprising a solvent and additionally comprising one or more stabilizers. In various embodiments, the solvent is an organic solvent. In various embodiments the solvent is an inorganic solvent.

In various embodiments, the solvent comprises acetaldehyde, acetone, acetonitrile, acetic acid, 1,2-butanediol, 1,3-butanediol, 1,4-butanedoil, 2-butoxyethanol, diethanolamine, diethylenetriamine, dimethoxyethane, dimethylformamide, 1,1-dimethylformamide, 1,1-dimethylhydrazine, 1,2-dimethylhydrazine, dimethyl sulfoxide, 1,4-dioxane, formic acid, ethanol, ethyl acetate, ethylamine, ethylene glycol, furfuryl alcohol, glycerol, isopropanol, methanol, methyl diethanolamine, methyl isocyanide, N-methyl-2-pyrrolidone, 1-propanol, 1,3-propanediol, 1,5-pentanediol, 2-propanol, propylene glycol, pyridine, tetrahydrofuran, triethylene glycol, benzoic acid, ascorbic acid, trichloroacetic acid, dichloroacetic acid, chloroacetic acid, trifluoroacetic acid, fluoroacetic acid, tartaric acid, lactic acid, gluconic acid, methanesulfonic acid, ethanesulfonic acid, benzenesulfonic acid, p-toluenesulfonic acid, trifluoromethanesulfonic acid, polystyrene sulfonic acid, oxalic acid, carbonic acid, methylamine, diethylamine, pyridinesulfuric acid, hydrochloric acid, nitric acid, hydrobromic, hydroiodic acid, hypochlorous acid, chloric acid, chloric acid, perchloric acid, fluorosulfuric acid, fluoroantimonic acid, fluoroboric acid, hexafluorophosphoric acid, chromic acid, phosphoric acid, hydrofluoric acid, boric acid, barium hydroxide, calcium hydroxide, chromium hydroxide, potassium hydroxide, ammonium hydroxide, zinc hydroxide, sodium bicarbonate, sodium hydroxide, magnesium hydroxide, ammonium bicarbonate, ammonia, aluminium hydroxide, sodium carbonate, magnesium hydroxide, zinc hydroxide, ferrous hydroxide, lithium hydroxide, rubidium hydroxide. In various embodiments the one or more PBC associated antigens are dissolved in a solvent that comprises one or more acids and/or one or more bases.

In various embodiments the acid comprises acetic acid, sulfuric acid, hydrochloric acid, nitric acid, formic acid, benzoic acid, ascorbic acid, trichloroacetic acid, dichloroacetic acid, chloroacetic acid, trifluoroacetic acid, fluoroacetic acid, tartaric acid, lactic acid, gluconic acid, methanesulfonic acid, ethanesulfonic acid, benzenesulfonic acid, p-toluenesulfonic acid, trifluoromethanesulfonic acid, polystyrene sulfonic acid, hydrobromic acid, hydroiodic acid, hypochlorous acid, chloric acid, chloric acid, perchloric acid, fluorosulfuric acid, fluoroantimonic acid, fluoroboric acid, hexafluorophosphoric acid, chromic acid, phosphoric acid, hydrofluoric acid, oxalic acid, boric acid, carbonic acid.

In various embodiments, the base comprises barium hydroxide, calcium hydroxide, chromium hydroxide, potassium hydroxide, ammonium hydroxide, zinc hydroxide, barium hydroxide, sodium bicarbonate, methylamine, diethylamine, sodium hydroxide, magnesium hydroxide, ammonium bicarbonate, ammonia aluminium hydroxide, sodium carbonate, magnesium hydroxide, zinc hydroxide, ferrous hydroxide, acetone, lithium hydroxide, pyridine, rubidium hydroxide.

In various embodiments, the concentration of the acid is between 0.1N to 36N including all ranges and values lying within this range. In various embodiments the concentration is about 0.1N, about 0.5N, about 1N, about 2N, about 3N, about 4N, about 5N, about 6N, about 7N, about 8N, about 9N, about 10N, about 11N, about 12N, about 13N, about 14N, about 15N, about 16N, about 17N, about 18N, about 20N, about 30N, or about 36N including all values lying within this range. In various embodiments, the concentration of the base is between 0.01% to 100% (v/v or wt/v) including all values lying within this range. In various embodiments the concentration is about 0.01%, about 0.05%, about 0.1%, about 0.2%, about 0.3%, about 0.4%, about 0.5%, about 0.6%, about 0.7%, about 0.8%, about 0.9%, about 1%, about 10%, about 25%, about 50%, about 75%, or about 100% (v/v or wt/v).

In various embodiments the acid has a pH between pH 1.0 to pH 7.0 including all ranges and values lying within this range. In various embodiments the pH is about pH 1.0, about pH 1.5, about pH 2.0, about pH 2.5, about pH 3.0, about pH 3.5, about pH 4.0, about pH 4.5, about pH 5.0, about pH 5.5, about pH 6.0, about pH 6.5, or about pH 7.0. In various embodiments, the base has a pH between pH 7.0 to pH 14.0 including all ranges and values lying within this range. In various embodiments the base pH is about pH 7.5, about pH 8.0, about pH 8.5, about pH 9.0, about pH 9.5, about pH 10.0, about pH 10.5, about pH 11.0, about pH 11.5, about pH 12.0, about pH 12.5, about pH 13.0, about pH 13.5, or about pH 14.0.

In various embodiments, one or more PBC associated antigens in step (a) are dissolved in an aqueous solution comprising one or more stabilizers. In various embodiments, the stabilizers comprise detergents/surfactants, osmolytes, metal complexes, proteins or amino acids. In various embodiments, the stabilizers are selected from the group comprising poly vinyl alcohol, poly(acrylic acid) (PAA), sorbitan monostearate, TritonX, Triton X-100, poloxamer, polyvinylpyrrolidone, Pluronics F68, n-dodecyl-β-D-maltoside (DDM), lauryl maltose neopentyl glycol (LMNG), 1-myristoyl-2-hydroxy-sn-glycero-3-[phospho-rac-(1-glycerol)] (LMPG), 1-palmitoyl-2-hydroxy-sn-glycero-3-[phospho-rac-(1-glycerol)] (LPPG), polyethylene glycol 400 dedecyl ether (Thesit), nonylphenyl polyethylene glycol (NP40), polyoxyethylene-(10)-dodecyl-ether (Genapol C-100), dodecyl-phosphocholine (DPC), n-decyl-β-maltoside (DM), 1,2-dioctanoyl-sn-glycero-3-phosphocholine (diC₈PC), 1,2-dihexanoyl-sn-glycero-3-phosphocholine (diC₆PC), 1,2-diheptanoyl-sn-glycero-3-phosphocholine (DHPC), n-octyl-β-D-glucose (B-OG), Brih-35, Brij-56, Brij-58, Brij-72, Brij-78, Brij-97, Brij-98, 3-((3-cholamidopropyl) dimethylammonio)-1-propanesulfonate) (CHAPS), disulphile containing detergents, fluorinated diglucose detergents, fluorinated maltose detergents, 1,3,5-triazine-cored maltoside amphiphiles, steroid based pentasaccharides, Vitamin-E based glycoside amphiphiles, calixarene based detergents, cyclodextrin, trehalose, mannitol, dextran, carboxymethyl cellulose, ethyl stearate, sodium acetate, sodium glutamate, polysorbate, albumin, bovine serum albumin, lysine, histidine, arginine, zinc salts, sodium bicarbonate, or magnesium hydroxide. In various embodiments the concentration of the stabilizer in step (a) is between 0.01% and 15% (e.g. about 0.01%, 0.05%, 0.1%, 0.5%, 1%, 5%, 10% or 15%) including all values lying within this range.

In various embodiments, the one or more PBC-associated antigens are dissolved in the solvent by mixing for between 0.1 to 96 hours including all including all values lying within this range. In various embodiments, the one or more PBC-associated antigens are dissolved in the solvent by mixing for about 0.1, about 0.2, about 0.3, about 0.4, about 0.5, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 48, about 72, or about 96 hours.

In various embodiments, the one or more PBC associated antigens are manufactured synthetically. In various embodiments, the antigens are manufactured by solid phase peptide synthesis or solution phase peptide synthesis. In various embodiments, the PBC associated antigens are manufactured by recombinant protein production technology, e.g., in mammalian cells, E. coli or other bacterial cells, yeast, insects, or in a cell free system.

In various embodiments the pH of the solution with one or more PBC associated antigens dissolved in step (a) is adjusted with a solvent.

In various embodiments the solvent comprises one or more acids and/or one or more base. In various embodiments the solvent has a pH between pH 1.0 to pH 14.0 including all values lying between this range. In various embodiments the solvent has a pH between pH 0.5 to pH 14.0 including all values lying between this range. In various embodiments the pH is about pH 1.0, about pH 1.5, about pH 2.0, about pH 2.5, about pH 3.0, about pH 3.5, about pH 4.0, about pH 4.5, about pH 5.0, about pH 5.5, about pH 6.0, about pH 6.5, about pH 7.0, about pH 7.5, about pH 8.0, about pH 8.5, about pH 9.0, about pH 9.5, about pH 10.0, about pH 10.5, about pH 11.0, about pH 11.5, about pH 12.0, about pH 12.5, about pH 13.0, about pH 13.5, or about pH 14.0. In various embodiments the pH is about pH 0.9. In various embodiments the solvent comprising acid is acetic acid, sulfuric acid, hydrochloric acid, nitric acid, formic acid, benzoic acid, ascorbic acid, trichloroacetic acid, dichloroacetic acid, chloroacetic acid, trifluoroacetic acid, fluoroacetic acid, tartaric acid, lactic acid, gluconic acid, methanesulfonic acid, ethanesulfonic acid, benzenesulfonic acid, p-toluenesulfonic acid, trifluoromethanesulfonic acid, polystyrene sulfonic acid, hydrobromic, hydroiodic acid, hypochlorous acid, chloric acid, chloric acid, perchloric acid, fluorosulfuric acid, fluoroantimonic acid, fluoroboric acid, hexafluorophosphoric acid, chromic acid, phosphoric acid, hydrofluoric acid, oxalic acid, boric acid, and carbonic acid. In various embodiments, the solvent comprising base is barium hydroxide, calcium hydroxide, chromium hydroxide, potassium hydroxide, ammonium hydroxide, zinc hydroxide, barium hydroxide, sodium bicarbonate, methylamine, diethylamine, sodium hydroxide, magnesium hydroxide, ammonium bicarbonate, ammonia aluminum hydroxide, sodium carbonate, magnesium hydroxide, zinc hydroxide, ferrous hydroxide, acetone, lithium hydroxide, pyridine, rubidium hydroxide. In various embodiments the solvent concentration is between 0.1 N to 36 N including all values lying within this range. In various embodiments the acid concentration is about 0.1N, about 0.5N, about 1N, about 2N, about 3N, about 4N, about 5N, about 6N, about 7N, about 8N, about 9N, about 10N, about 11N, about 12N, about 13N, about 14N, about 15N, about 16N, about 17N, about 18N, about 20N, about 30N, about 36N including all values lying within this range. In various embodiments, the concentration of the solvent is between 0.01% and 100% (v/v or wt/v) including all values lying within this range. In various embodiments the concentration of the solvent is about 0.01%, about 0.05%, about 0.1%, about 0.2%, about 0.3%, about 0.4%, about 0.5%, about 0.6%, about 0.7%, about 0.8%, about 0.9%, about 1%, about 10%, about 25%, about 50%, about 75%, or about 100% (v/v or wt/v) including all values lying within this range. In various embodiments the adjusted pH of the solution with dissolved PBC associated antigens is between pH 1.0 to pH 7.0. In various embodiments the pH is about pH 1.0, about pH 1.5, about pH 2.0, about pH 2.5, about pH 3.0, about pH 3.5, about pH 4.0, about pH 4.5, about pH 5.0, about pH 5.5, about pH 6.0, about pH 6.5, about pH 7.0 including all ranges and values lying within this range. In various embodiments, the adjusted pH of the solution with dissolved PBC associated antigens is between pH 6.0 to pH 7.5. In various embodiments, the pH of the solution with one or more PBC associated antigens dissolved in step (a) is adjusted by dialysis. In various embodiments, the pH is adjusted prior to, during, or after the generation of the primary emulsion in step (b).

In various embodiments, the PDC-E2 antigen in step (a) is dissolved in an aqueous solution that comprises one or more acids and/or one or more bases.

In various embodiments, the PDC-E2 antigen in step (a) is dissolved in an aqueous solution comprising a base such as ammonium hydroxide, barium hydroxide, calcium hydroxide, chromium hydroxide, potassium hydroxide, zinc hydroxide, barium hydroxide, sodium bicarbonate, methylamine, diethylamine, sodium hydroxide, magnesium hydroxide, ammonium bicarbonate, ammonia aluminium hydroxide, sodium carbonate, magnesium hydroxide, zinc hydroxide, ferrous hydroxide, acetone, lithium hydroxide, pyridine, rubidium hydroxide. Once the dissolved, the pH can be adjusted with an acid such as acetic acid, sulfuric acid, hydrochloric acid, nitric acid, formic acid, benzoic acid, ascorbic acid, trichloroacetic acid, dichloroacetic acid, chloroacetic acid, trifluoroacetic acid, fluoroacetic acid, tartaric acid, lactic acid, gluconic acid, methanesulfonic acid, ethanesulfonic acid, benzenesulfonic acid, p-toluenesulfonic acid, trifluoromethanesulfonic acid, polystyrene sulfonic acid, hydrobromic acid, hydroiodic acid, hypochlorous acid, chloric acid, chloric acid, perchloric acid, fluorosulfuric acid, fluoroantimonic acid, fluoroboric acid, hexafluorophosphoric acid, chromic acid, phosphoric acid, hydrofluoric acid, oxalic acid, boric acid, carbonic acid. In one embodiment, PDC-E2 is dissolved in an aqueous ammonium hydroxide solution (e.g. a 0.1% ammonium hydroxide solution). Once PDC-E2 is dissolved, the pH can be adjusted with an acid such as acetic acid (e.g. 0.9 N or 1N acetic acid).

In various embodiments the aqueous solution is diluted to the desired concentration in step (a) using a solvent. In various embodiments, the solution is diluted prior to, during, or after the generation of the primary emulsion in step (b). In various embodiments, the solvent for dilution comprises an organic solvent. In various embodiments, the solvent comprises an inorganic solvent. In various embodiments, the solvent comprises water.

In various embodiments, the concentration of the one or more PBC associated antigens dissolved in step (a) is between about 0.1 mg/mL to about 100 mg/mL including all values lying within this range. In various embodiments, the concentration of the dissolved one or more PBC associated antigens is between about 1 mg/mL to about 10 mg/mL. In various embodiments, the concentration of the dissolved one or more PBC associated antigens is about 0.1 mg/mL, about 0.2 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL.

In various embodiments, the emulsion of step (b) includes a solvent. In various embodiments, the solvent comprises an organic solvent. In various embodiments, the solvent comprises an inorganic solvent. In various embodiments, the organic solvent is selected from the group consisting of acetone, methylene chloride (dichloromethane), dimethyl sulfoxide (DMSO), ethyl acetate, dimethylformamide, tetrahydrofuran, chloroform, and acetic acid. In various embodiments, the inorganic solvent is selected from the group consisting of water, ammonia, an aqueous ammonium hydroxide solution, sulphuric acid, carbon disulphide, bromine trifluoride, phosphorous oxychloride, hydrogen fluoride, and sulphur dioxide. In various embodiments, the solvent of step (b) is at a concentration between 1% (v/v) to 50% (v/v) including all values within this range. In various embodiments, the solvent of step (b) is at a concentration between 0.1% (v/v) to 50% (v/v) including all values within this range. In various embodiments, the solvent of step (b) is at a concentration of about 1%, about 2%, about 3%, about 5%, about 10%, about 15%, about 20%, about 25%, about 30%, about 35%, about 40%, about 45%, or about 50% (v/v). In various embodiments, the solvent of step (b) is at a concentration between 0.1 mM to 10 mM including all values lying between this range. In various embodiments, the solvent of step (b) is at a concentration of about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7.0 mM, about 7.5 mM, about 8.0 mM, about 8.5 mM, about 9.0 mM, or about 10.0 mM. In various embodiments, the solvent of step (b) is at a concentration between 0.1 M to 10 M including all values lying between this range. In various embodiments, the solvent of step (b) is at a concentration of about 0.1 M, about 0.2 M, about 0.3 M, about 0.4 M, about 0.5 M, about 0.6 M, about 0.7 M, about 0.8 M, about 0.9 M, about 1.0 M, about 1.5 M, about 2 M, about 2.5 M, about 3 M, about 3.5 M, about 4 M, about 4.5 M, about 5 M, about 5.5 M, about 6 M, about 6.5 M, about 7.0 M, about 7.5 M, about 8.0 M, about 8.5 M, about 9.0 M, or about 10.0 M, including all values lying between this range.

In various embodiments, the polymer in step (b) comprises a biodegradable polymer. In various embodiments, the biodegradable polymer comprises polyglycolic acid (PGA), polylactic acid (PLA), polysebacic acid (PSA), poly(lactic-co-glycolic) (PLGA), poly(lactic-co-sebacic) acid (PLSA), poly(glycolic-co-sebacic) acid (PGSA), polypropylene sulfide, poly(caprolactone), chitosan, a polysaccharide, or a lipid. In various embodiments, the polymer is a co-polymer. In various embodiments, the co-polymer has varying molar ratios of constituent polymers. In various embodiments, the molar ratio is 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0 including all values lying within this range.

In various embodiments, the polymer in step (b) comprises PLGA. In various embodiments, the molar ratio of co-polymers of PLGA are 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0 including all values lying within this range. In various embodiments, the PLGA has a high molecular weight. In various embodiments, the PLGA has a low molecular weight. In various embodiments, the PLGA has a molecular weight of between 1 kDa and 100 kDa (e.g., between 1 kDa, 5 kDa, 10 kDa, 20 kDa, 30 kDa, 40 kDa, 50 kDa, 60 kDa, 70 kDa, 80 kDa, 90 kDa, 100 kDa) including all values lying within this range. In various embodiments, the amount of PLGA in step (b) is between 0.05 and 100% (e.g., between 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight. In various embodiments, the polymer in step (b) is dissolved in an organic solvent. In various embodiments, the polymer in step (b) is dissolved in methylene chloride (dichloromethane), ethyl acetate, dimethylformamide, tetrahydrofuran, or chloroform. In various embodiments, the PLGA polymer is dissolved in ethyl acetate. In various embodiments, the method comprises 5% PLGA solution (50:50) having a molecular weight between 10,000 to 60,000 Daltons (Da).

In various embodiments, the surfactant and/or stabilizer mixture of step (c) includes a solvent. In various embodiments, the solvent comprises an organic solvent. In various embodiments, the solvent comprises an inorganic solvent. In various embodiments, the organic solvent is selected from the group consisting of acetone, ethanol, methylene chloride (dichloromethane), dimethyl sulfoxide (DMSO), ethyl acetate, dimethylformamide, tetrahydrofuran, chloroform, and acetic acid. In various embodiments, the inorganic solvent is selected from the group consisting of water, ammonia, sulphuric acid, carbon disulphide, bromine trifluoride, phosphorous oxychloride, hydrogen fluoride, and sulphur dioxide. In various embodiments, the solvent in the mixture is at a concentration between 1% (v/v) to 50% (v/v) including all values within this range. In various embodiments, the solvent in the mixture is at a concentration of about 1%, about 2%, about 3%, about 5%, about 10%, about 15%, about 20%, about 25%, about 30%, about 35%, about 40%, about 45%, or about 50% (v/v). In various embodiments, the solvent in the mixture is at a concentration between 0.1 mM to 10 mM including all values lying between this range. In various embodiments, the solvent in the mixture is at a concentration of about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, about 1.5 mM, about 2 mM, about 2.5 mM, about 3 mM, about 3.5 mM, about 4 mM, about 4.5 mM, about 5 mM, about 5.5 mM, about 6 mM, about 6.5 mM, about 7.0 mM, about 7.5 mM, about 8.0 mM, about 8.5 mM, about 9.0 mM, or about 10.0 mM. In various embodiments, the solvent in the mixture is at a concentration between 0.1 M to 10 M including all values lying between this range. In various embodiments, the solvent in the mixture is at a concentration of about 0.1 M, about 0.2 M, about 0.3 M, about 0.4 M, about 0.5 M, about 0.6 M, about 0.7 M, about 0.8 M, about 0.9 M, about 1.0 M, about 1.5 M, about 2 M, about 2.5 M, about 3 M, about 3.5 M, about 4 M, about 4.5 M, about 5 M, about 5.5 M, about 6 M, about 6.5 M, about 7.0 M, about 7.5 M, about 8.0 M, about 8.5 M, about 9.0 M, or about 10.0 M. In various embodiments, the solvent in step (b) and step (c) are the same. In various embodiments, the solvent in step (b) and step (c) are different.

In various embodiments, the emulsion of step (b) includes between 0.001 mg/mL to 10 mg/mL of one or more PBC associated antigens including all values lying within this range. In various embodiments, the emulsion of step (b) includes about 0.0011 mg/mL, about 0.002 mg/mL, about 0.005 mg/mL, about 0.01 mg/mL, about 0.02 mg/mL, about 0.03 mg/mL, about 0.04 mg/mL, about 0.05 mg/mL, about 0.06 mg/mL, about 0.07 mg/mL, about 0.08 mg/mL, about 0.09 mg/mL, about 0.1 mg/mL, about 0.2 mg/mL, about 0.3 mg/mL, about 0.4 mg/mL, about 0.5 mg/mL, about 1 mg/mL, about 2 mg/mL, about 3 mg/mL, about 4 mg/mL, about 5 about 6 mg/mL, about 7 mg/mL, about 8 mg/mL, or about 10 mg/mL of the one or more PBC associated antigens.

In various embodiments, the emulsion of step (b) includes between 0.1 mg/mL to 100 mg/mL of one or more PBC associated antigens including all values lying within this range. In various embodiments, the emulsion of step (b) includes about 0.1 mg/mL, about 0.2 mg/mL, about 0.5 mg/mL, about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL of one or more PBC associated antigens.

In some embodiments, the emulsion of step (b) includes between 0.2 μg of PBC associated antigen/mg polymer to 100 μg of PBC associated antigen/mg polymer including all values lying within this range. In some embodiments, the emulsion of step (b) includes 0.2 μg/mg, 2 μg/mg, 5 μg/mg, 10 μg/mg, 20 μg/mg, 25 μg/mg, 50 μg/mg, 100 μg/mg, 200 μg/mg including all values lying within this range.

In various embodiments, the surfactant and/or stabilizer used in step (c) is anionic, cationic, nonionic or zwitterionic. In various embodiments, the surfactant and/or stabilizer is a poloxamer, a polyamine, polyethylene glycol (PEG), Tween-80, gelatin, dextran, pluronic L-63, pluronic F-68, pluronic 188, pluronic F-127, polyvinyl alcohol (PVA), polyacrylic acid (PAA), methylcellulose, lecithin, didodecyldimethylammonium bromide (DMAB), poly(ethylene-alt-maleic acid) (PEMA), vitamin E TPGS (D-a-tocopheryl polyethylene glycol 1000 succinate), hyaluronic acid, poly amino acids (e.g., polymers of lysine, arginine, aspartic acid, glutamic acid, serine, threonine, tyrosine and cysteine, or their enantiomers), methylcellulose, hydroxyethylcellulose, hydroxyprolylcellulose, hydroxypropylmethylcellulose, gelatin, sodium cholate, a carbomer, or a sulfate polymer (e.g., heparin sulfate, chondroitin sulfate, fucoidan, ulvan, and carrageenan). In various embodiments, the amount of surfactant and/or stabilizer present in the mixture in step (c) is between 0.05 and 100% (e.g., between 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight or volume. In various embodiments, the surfactant and/or stabilizer have a molecular weight of between 0.1 to 10,000 kDa (e.g., between 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 500, 1000, 2000, 3000, 4000, 5000, 6000, 7000, 8000, 9000, or 10000 kDa including all values lying within this range). In various embodiments, the mixture is prepared by admixing 4% PVA and PAA (100 kDa, 35% wt) with ethyl acetate. In various embodiments, the mixture is prepared by admixing 4% PVA in water and PAA (100 kDa, 35% wt) in water with ethyl acetate.

In various embodiments, the solution or mixture including one or more surfactants and/or stabilizers that form an oil-in-water secondary emulsion in (c) has a pH less than pH 4.0. In various embodiments, the oil-in-water secondary emulsion has a pH between pH 1.0 and less than pH 4.0 including all values lying within this range. In various embodiments, the oil-in-water secondary emulsion has a pH of about pH 1.0 to pH less than pH 4.0, about pH 2.0 to pH less than pH 4, about pH 3 to pH less than pH 4, about pH 1.0 to about pH 3.5, about pH 2.0 to about pH 3.5, about pH 3 to about pH 3.5, about pH 1, about pH 1.5, about pH 2, about pH 2.5, about pH 3, about pH 3.5, about pH 3.6, about pH 3.7, about pH 3.8 or about pH 3.9, including all values lying within this range.

In various embodiments, the water-in-oil primary emulsion of step (b) is obtained by homogenization of the aqueous solution of one or more PBC associated antigens with the oil phase including a polymer. In various embodiments, homogenization is performed between 5 seconds to 1000 seconds including all values lying within this range. In various embodiments, homogenization is performed for about 5, about 10, about 15, about 20, about 25, about 30, about 35, about 40, about 45, about 60, about 90, about 120, about 150, about 180, about 210, about 240, about 270, about 300, about 330, about 360, about 390, about 420, about 450, about 480, about 510, about 540, about 570, about 600, about 700, about 800, about 900, or about 1000 seconds. In various embodiments, the oil-in-water secondary emulsion of step (c) is obtained by homogenization of the primary emulsion with a solution including one or more surfactants and/or stabilizer. In various embodiments, homogenization is performed between 5 seconds to 1000 seconds including all values lying within this range. In various embodiments, homogenization is performed for about 5, about 10, about 15, about 20, about 25, about 30, about 35, about 40, about 45, about 60, about 90, about 120, about 150, about 180, about 210, about 240, about 270, about 300, about 330, about 360, about 390, about 420, about 450, about 480, about 510, about 540, about 570, about 600, about 700, about 800, about 900, or about 1000 seconds. In various embodiments, the water-in-oil primary emulsion of step (b) is obtained by sonication of the aqueous solution of PBC associated antigens with the oil phase including a polymer. In various embodiments, sonication is performed between 5 seconds to 1000 seconds including all values lying within this range. In various embodiments, sonication is performed for about 5, about 10, about 15, about 20, about 25, about 30, about 35, about 40, about 45, about 60, about 90, about 120, about 150, about 180, about 210, about 240, about 270, about 300, about 330, about 360, about 390, about 420, about 450, about 480, about 510, about 540, about 570, about 600, about 700, about 800, about 900, or about 1000 seconds. In various embodiments, the oil-in-water secondary emulsion of step (c) is obtained by sonication of the primary emulsion of step (b) with a mixture including one or more surfactants and/or stabilizers. In various embodiments, sonication is performed between 5 seconds to 1000 seconds including all values lying within this range. In various embodiments, sonication is performed for about 5, about 10, about 15, about 20, about 25, about 30, about 35, about 40, about 45, about 60, about 90, about 120, about 150, about 180, about 210, about 240, about 270, about 300, about 330, about 360, about 390, about 420, about 450, about 480, about 510, about 540, about 570, about 600, about 700, about 800, about 900, or about 1000 seconds.

In various embodiments, the secondary emulsion is hardened by evaporation. In various embodiments, the evaporation is active evaporation. In various embodiments, the active evaporation is performed using stirring or under vacuum i.e. vacuum-driven evaporation. In various embodiments, the active evaporation is performed under high-pressure vacuum. In various embodiments, the active evaporation is performed under low pressure vacuum. In various embodiments, the evaporation is passive evaporation. In various embodiments, evaporation is performed between 0.25 hours and 96 hours including all values lying within this range. In various embodiments, evaporation is performed for about 0.5, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 48, about 72, or about 96 hours.

In various embodiments, the evaporation is performed at a pressure of between 0.01 and 1000 mBar (e.g., between 0.01, 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, or 1000 mBar including all including all values lying within this range).

In various embodiments, the filtration, washing, and concentration of particles in step (e) is performed by gel filtration, membrane filtration, dialysis, centrifugation, chromatography, density gradient centrifugation, tangential flow filtration (TFF) or combinations thereof.

In various embodiments, the disclosure further contemplates a method for manufacturing a composition comprising negatively charged particles encapsulating PDC-E2₁₅₅₋₁₈₅ peptides comprising amino acid sequence KVGEKLSEGDLLAEIETDKATIGFEVQEEGY (SEQ ID NO: 1) comprising: (a) generating an aqueous solution of PDC-E2₁₅₅₋₁₈₅ peptides; (b) generating a primary emulsion by homogenizing the aqueous solution of (a) with an oil phase including poly(lactic-co-glycolic) (PLGA) dissolved in ethyl acetate; (c) homogenizing the primary emulsion of step (b) with a mixture comprising ethyl acetate, polyacrylic acid (PAA) and polyvinyl alcohol (PVA) to form a secondary emulsion; (d) hardening the secondary emulsion by evaporation resulting in hardened polymeric nanoparticles encapsulating PDC-E2₁₅₅₋₁₈₅ peptides within their cores.

In various embodiments, the disclosure further contemplates a method for manufacturing a composition comprising negatively charged particles encapsulating PDC-E2₁₅₅₋₁₈₅ peptides comprising amino acid sequence KVGEKLSEGDLLAEIETDKATIGFEVQEEGY (SEQ ID NO: 1) comprising: (a) generating an aqueous solution of PDC-E2₁₅₅₋₁₈₅ peptides; (b) generating a primary emulsion by homogenizing the aqueous solution of (a) with an oil phase including poly(lactic-co-glycolic) (PLGA) dissolved in ethyl acetate; (c) homogenizing the primary emulsion of step (b) with a mixture comprising ethyl acetate, polyacrylic acid (PAA) and polyvinyl alcohol (PVA) and water to form a secondary emulsion; (d) hardening the secondary emulsion by evaporation resulting in hardened polymeric nanoparticles encapsulating PDC-E2₁₅₅₋₁₈₅ peptides within their cores.

In various embodiments, the method further comprises (e) filtering, washing, and concentrating the nanoparticles. In various embodiments, the method further comprises (e) filtering, washing, and concentrating the nanoparticles and (f) addition of sodium citrate, mannitol and sucrose and freeze drying the nanoparticles.

In various embodiments, the disclosure provides a method for manufacturing a composition comprising negatively charged particles encapsulating PDC-E2₁₅₅₋₁₈₅ peptides comprising: (a) generating an aqueous solution of PDC-E2₁₅₅₋₁₈₅ peptides between pH 1.0 and pH 7.0; (b) generating a primary emulsion by homogenizing the aqueous solution of (a) with an oil phase including poly(lactic-co-glycolic) (PLGA) dissolved in ethyl acetate; (c) homogenizing the primary emulsion with a mixture comprising ethyl acetate, polyacrylic acid (PAA) and polyvinyl alcohol (PVA) to form a secondary emulsion; (d) hardening the secondary emulsion by evaporation resulting in hardened polymeric nanoparticles encapsulating PDC-E2₁₅₅₋₁₈₅ peptides within their cores; (e) filtering, washing, and concentrating the nanoparticles; and (f) addition of sodium citrate, mannitol and sucrose and freeze drying the nanoparticles. In various embodiments, the pH is between pH 6.0 and 7.0.

In various embodiments, the disclosure provides a method for manufacturing a composition comprising negatively charged particles encapsulating PDC-E2₁₅₅₋₁₈₅ peptides comprising: (a) generating an aqueous solution of PDC-E2₁₅₅₋₁₈₅ peptides between pH 1.0 and pH 7.0; (b) generating a primary emulsion by homogenizing the aqueous solution of (a) with an oil phase including poly(lactic-co-glycolic) (PLGA) dissolved in ethyl acetate; (c) homogenizing the primary emulsion with a mixture comprising ethyl acetate, polyacrylic acid (PAA), polyvinyl alcohol (PVA) and water to form a secondary emulsion; (d) hardening the secondary emulsion by evaporation resulting in hardened polymeric nanoparticles encapsulating PDC-E2₁₅₅₋₁₈₅ peptides within their cores; (e) filtering, washing, and concentrating the nanoparticles; and (f) addition of sodium citrate, mannitol and sucrose and freeze drying the nanoparticles. In various embodiments, the pH is between pH 6.0 and 7.0.

In various embodiments, the disclosure provides a method for manufacturing a composition comprising negatively charged particles encapsulating PDC-E2₁₅₅₋₁₈₅ peptides comprising the amino acid sequence KVGEKLSEGDLLAEIETDKATIGFEVQEEGY (SEQ ID NO: 1) comprising: (a) dissolving PDC-E2₁₅₅₋₁₈₅ peptides in ammonium hydroxide, adjusting pH to between about pH 1.0 and about pH 7.0 with acetic acid and diluting to about 5 mg/mL to 10 mg/ml peptide concentration with water (such as sterile water for injection (WFI)); (b) generating a primary emulsion by homogenizing the aqueous solution of (a) with an oil phase including poly(lactic-co-glycolic) (PLGA) dissolved in ethyl acetate; (c) homogenizing the primary emulsion of step (b) with a mixture comprising ethyl acetate, polyacrylic acid (PAA) and polyvinyl alcohol (PVA), and optionally water, to form a secondary emulsion; (d) hardening the secondary emulsion by evaporation resulting in hardened polymeric nanoparticles encapsulating PDC-E2₁₅₅₋₁₈₅ peptides within their cores.

In various embodiments, the disclosure further contemplates a method for manufacturing a composition comprising negatively charged particles encapsulating PDC-E2₁₅₅₋₁₈₅ peptides comprising the amino acid sequence KVGEKLSEGDLLAEIETDKATIGFEVQEEGY (SEQ ID NO: 1) comprising: (a) dissolving PDC-E2₁₅₅₋₁₈₅ peptides in ammonium hydroxide, adjusting pH to between about pH 1.0 and about pH 7.0 with acetic acid and diluting to approximately 8.25 mg/mL peptide concentration with water (such as sterile water for injection (WFI); (b) generating a primary emulsion by homogenizing the aqueous solution of (a) with an oil phase including poly(lactic-co-glycolic) (PLGA) dissolved in ethyl acetate; (c) homogenizing the primary emulsion of step (b) with a mixture comprising ethyl acetate, polyacrylic acid (PAA) and polyvinyl alcohol (PVA) to form a secondary emulsion; (d) hardening the secondary emulsion by evaporation resulting in hardened polymeric nanoparticles encapsulating PDC-E2₁₅₅₋₁₈₅ peptides within their cores.

In various embodiments, the disclosure further contemplates a method for manufacturing a composition comprising negatively charged particles encapsulating PDC-E2₁₅₅₋₁₈₅ peptides comprising the amino acid sequence KVGEKLSEGDLLAEIETDKATIGFEVQEEGY (SEQ ID NO: 1) comprising: (a) dissolving PDC-E2₁₅₅₋₁₈₅ peptides in ammonium hydroxide, adjusting pH to between about pH 1.0 and about pH 7.0 with acetic acid and diluting to approximately 8.25 mg/mL peptide concentration with water for injection (WFI); (b) generating a primary emulsion by homogenizing the aqueous solution of (a) with an oil phase including poly(lactic-co-glycolic) (PLGA) dissolved in ethyl acetate; (c) homogenizing the primary emulsion of step (b) with a mixture comprising ethyl acetate, polyacrylic acid (PAA) and polyvinyl alcohol (PVA) and water to form a secondary emulsion; (d) hardening the secondary emulsion by evaporation resulting in hardened polymeric nanoparticles encapsulating PDC-E2₁₅₅₋₁₈₅ peptides within their cores.

In various embodiments, the method further comprises (e) filtering, washing, and concentrating the nanoparticles. In various embodiments, the method further comprises (e) filtering, washing, and concentrating the nanoparticles and (f) addition of sodium citrate, mannitol and sucrose and freeze drying the nanoparticles.

In various embodiments, the disclosure further contemplates a method for manufacturing a composition comprising negatively charged particles encapsulating PDC-E2₁₅₅₋₁₈₅ peptides comprising the amino acid sequence KVGEKLSEGDLLAEIETDKATIGFEVQEEGY (SEQ ID NO: 1) comprising: (a) dissolving PDC-E2₁₅₅₋₁₈₅ peptides in ammonium hydroxide, adjusting pH to between about pH 1.0 and about pH 7.0 with acetic acid and diluting to approximately 8.25 mg/mL peptide concentration with water (such as sterile water for injection (WFI)); (b) generating a primary emulsion by homogenizing the aqueous solution of (a) with an oil phase including poly(lactic-co-glycolic) (PLGA) dissolved in ethyl acetate; (c) homogenizing the primary emulsion with a mixture comprising ethyl acetate, polyacrylic acid (PAA) and polyvinyl alcohol (PVA) to form a secondary emulsion; (d) hardening the secondary emulsion by evaporation resulting in hardened polymeric nanoparticles encapsulating PDC-E2₁₅₅₋₁₈₅ peptides within their cores; (c) filtering, washing, and concentrating the nanoparticles; and (f) addition of sodium citrate, mannitol and sucrose and freeze drying the nanoparticles.

In various embodiments, the disclosure further contemplates a method for manufacturing a composition comprising negatively charged particles encapsulating PDC-E2₁₅₅₋₁₈₅ peptides comprising the amino acid sequence KVGEKLSEGDLLAEIETDKATIGFEVQEEGY (SEQ ID NO: 1) comprising: (a) dissolving PDC-E2₁₅₅₋₁₈₅ peptides in ammonium hydroxide, adjusting pH to between about pH 1.0 and about pH 7.0 with acetic acid and diluting to approximately 8.25 mg/mL peptide concentration with water (such as WFI); (b) generating a primary emulsion by homogenizing the aqueous solution of (a) with an oil phase including poly(lactic-co-glycolic) (PLGA) dissolved in ethyl acetate; (c) homogenizing the primary emulsion with a mixture comprising ethyl acetate, polyacrylic acid (PAA) and polyvinyl alcohol (PVA) and water to form a secondary emulsion; (d) hardening the secondary emulsion by evaporation resulting in hardened polymeric nanoparticles encapsulating PDC-E2₁₅₅₋₁₈₅ peptides within their cores; (e) filtering, washing, and concentrating the nanoparticles; and (f) addition of sodium citrate, mannitol and sucrose and freeze drying the nanoparticles.

In various embodiments, the ammonium hydroxide is between about 0.05% to about 0.5%. In various embodiments, the ammonium hydroxide is 0.1%.

In various embodiments, the acetic acid is between about 0.5N to about 2N. In various embodiments, the acetic acid is 1 N acetic acid. In various embodiments, the acetic acid is between about 0.5N to about 2N. In various embodiments, the acetic acid is 0.9 N acetic acid.

In various embodiments, the disclosure further contemplates a method for manufacturing a composition comprising negatively charged particles encapsulating PDC-E2₁₅₅₋₁₈₅ peptides comprising: (a) dissolving PDC-E2₁₅₅₋₁₈₅ peptide in ammonium hydroxide, adjusting pH by dialysis to between about pH 6.0 and about pH 7.0; (b) generating a primary emulsion by homogenizing the aqueous solution of (a) with an oil phase including poly(lactic-co-glycolic) (PLGA) dissolved in ethyl acetate; (c) homogenizing the primary emulsion with a mixture comprising ethyl acetate, polyacrylic acid (PAA) and polyvinyl alcohol (PVA) to form a secondary emulsion; (d) hardening the secondary emulsion by evaporation resulting in hardened polymeric nanoparticles encapsulating PDC-E2₁₅₅₋₁₈₅ peptides within their cores; (c) filtering, washing, and concentrating the nanoparticles; and (f) addition of sodium citrate, mannitol and sucrose and freeze drying the nanoparticles. In various embodiments, the PDC-E2₁₅₅₋₁₈₅ peptides in step (a) are diluted to between about 5 mg/ml and about 10 mg/ml, e.g., approximately 8.25 mg/mL.

In various embodiments, the disclosure further contemplates a method for manufacturing a composition comprising negatively charged particles encapsulating PDC-E2₁₅₅₋₁₈₅ peptides comprising: (a) dissolving PDC-E2₁₅₅₋₁₈₅ peptide in ammonium hydroxide, adjusting pH by dialysis to between about pH 6.0 and about pH 7.0; (b) generating a primary emulsion by homogenizing the aqueous solution of (a) with an oil phase including poly(lactic-co-glycolic) (PLGA) dissolved in ethyl acetate; (c) homogenizing the primary emulsion with a mixture comprising ethyl acetate, polyacrylic acid (PAA) and polyvinyl alcohol (PVA) and water to form a secondary emulsion; (d) hardening the secondary emulsion by evaporation resulting in hardened polymeric nanoparticles encapsulating PDC-E2₁₅₅₋₁₈₅ peptides within their cores; (e) filtering, washing, and concentrating the nanoparticles; and (f) addition of sodium citrate, mannitol and sucrose and freeze drying the nanoparticles. In various embodiments, the PDC-E2₁₅₅₋₁₈₅ peptides in step (a) are diluted to between about 5 mg/ml and about 10 mg/ml, e.g., approximately 8.25 mg/mL.

In various embodiments, the method comprises a 5% PLGA solution comprising 50:50 PLGA having a molecular weight between 10,000 to 60,000 Da.

In various embodiments, the mixture comprising ethyl acetate, PAA, and PVA of (c) is prepared by adding 4% PVA and PAA (100 kDa, 35% wt) to ethyl acetate. In various embodiments, the PVA/PAA/ethyl acetate mixture is maintained at a pH below pH 4.0, below pH 3.9, below pH 3.8, below pH 3.7, below pH 3.6, or below pH 3.5.

In various embodiments, the mixture comprising ethyl acetate, PAA, and PVA and water of (c) is prepared by adding 4% PVA and PAA (100 kDa, 35% wt) to ethyl acetate. In various embodiments, the PVA/PAA/ethyl acetate/water mixture is maintained at a pH below pH 4.0, below pH 3.9, below pH 3.8, below pH 3.7, below pH 3.6, or below pH 3.5.

The present disclosure also contemplates a process for manufacturing a composition comprising negatively charged TIMPs encapsulating one or more PBC associated antigens (TIMP-PBC). In various embodiments, TIMP-PBC particles have a negative zeta potential. In various embodiments, the negative zeta potential of TIMP-PBC particles is between about −100 mV to about 0 mV. In various embodiments, the zeta potential of the particles is from about −100 mV to about −25 mV, from about −100 to about −30 mV, from about −80 mV to about −30 mV, from about −75 mV to about −30 mV, from about −70 mV to about −30 mV, from about −75 to about −35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV, including all values lying within this range.

In various embodiments, the size, or diameter, of TIMP-PBC particles is between 0.05 μm to about 10 μm. In various embodiments, the diameter of TIMP-PBC particles is between 0.1 μm and about 10 μm. In various embodiments, the diameter of TIMP-PBC particles is between 0.1 μm and about 5 μm. In various embodiments, the diameter of TIMP-PBC particles is between 0.1 μm and about 3 μm. In various embodiments, the diameter of TIMP-PBC particles is between 0.3 μm and about 5 μm. In various embodiments, the diameter of TIMP-PBC particles is about 0.3 μm to about 3 μm. In various embodiments, the diameter of TIMP-PBC particles is between about 0.3 μm to about 1 μm. In various embodiments, the diameter of TIMP-PBC particles is between about 0.4 μm to about 1 μm. In various embodiments, the TIMP-PBC particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-PBC particles have a diameter of about 50 nm, about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, or about 2000 nm. In various embodiments, the diameter of the negatively charged particle is between 400 nm to 800 nm. In various embodiments, the polydispersity index (PDI) or heterogeneity index for particle size is between 0.01 and 1.0 (e.g., 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, and 1 including all values within the range). In various embodiments, TIMP-PBC particles have a size between about 400 to about 800 nm and a zeta potential between −30 mV and −80 mV.

In various embodiments, the particles have a homogenous size distribution. In various embodiments, the particles have a homogenous size distribution wherein at least 90% of the particles have a diameter of between 0.05 μm and about 10 μm, between 0.1 μm and about 10 μm, 0.1 μm and about 5 μm, 0.1 μm and about 3 μm, 0.3 μm and about 5 μm, 0.3 μm to about 3 μm. In various embodiments, the particles have a homogenous size distribution wherein at least 90% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-PBC particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the particles have a homogenous size distribution wherein at least 50% of the particles have a diameter of between about 0.05 μm and about 10 μm, about 0.1 μm and about 10 μm, about 0.1 μm and about 5 μm, about 0.1 μm and about 3 μm, about 0.3 μm and about 5 μm, and about 0.3 μm and about 3 μm. In various embodiments, the particles have a homogenous size distribution wherein at least 50% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-PBC particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm. In various embodiments, the particles have a homogenous size distribution wherein at least 10% of the particles have a diameter of between about 0.05 μm and about 10 μm, about 0.1 μm and about 10 μm, about 0.1 μm and about 5 μm, about 0.1 μm and about 3 μm, about 0.3 μm and about 5 μm, and about 0.3 μm and about 3 μm. In various embodiments, the particles have a homogenous size distribution wherein at least 10% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm. In various embodiments, the TIMP-PBC particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm.

In some embodiments, the particles have a D90 of less than about 1000 nm. In some embodiments, the particles have a D50 of about 400 nm to about 800 nm, including about 400 nm, about 420 nm, about 440 nm, about 460 nm, about 480 nm, about 500 nm, about 510 nm, about 520 nm, about 530 nm, about 540 nm, about 550 nm, about 560 nm, about 570 nm, about 580 nm, about 590 nm, about 600 nm, about 650 nm, about 700 nm, about 750 nm, or about 800 nm including any values or ranges there between. In some embodiments, the particles have a D10 of less than about 600 nm. In some embodiments, the particles have a D90 of about 600 nm to about 700 nm. In some embodiments, the particles have a D50 of about 550 nm to about 600 nm. In some embodiments, the particles have a D10 of about 500 nm to about 550 nm. In various embodiments, the particles have a D[4,3] less than 1000 nm. In various embodiments the particles have a D[4,3] between 400 nm and 800 nm.

In some embodiments, the particles have a spherical or round shape.

In various embodiments, the present disclosure provides a process for manufacturing a composition comprising negatively charged particles encapsulating one or more PBC associated antigens (TIMP-PBC). In various embodiments, the amount of one or more PBC associated antigens encapsulated within the TIMP-PBC composition is 0.1 μg/mg to 100 μg/mg. In various embodiments, the one or more PBC associated antigen content is between 0.1 to 100 μg/mg (e.g., 0.1, 0.5, 1, 1.5, 2, 2.5, 3, 3.5, 4, 4.5, 5, 10, 15, 20, 25, 30, 35, 40, 45, 50, 55, 60, 65, 70, 75, 80, 85, 90, 95, or 100 μg/mg) including all values and ranges that lie in between these values. In various embodiments, the process of making TIMP-PBC as described herein yields an encapsulation efficiency between 1-100% (e.g., 1, 2, 3, 4, 5, 6, 7, 8, 9, 10, 15, 20, 25, 30, 40, 50, 60, 70, 80, 90, or 100% including all values and ranges that lie between these values). The one or more PBC associated antigen content in the TIMP-PBC composition can be determined by methods described in the literature including ELISA, Mass Spectrometry, NMR, HPLC, CBQCA, and Western Blot.

In various embodiments, the present disclosure provides a process for manufacturing a composition comprising negatively charged particles encapsulating one or more PBC associated antigens (TIMP-PBC), wherein the particle surface contains low levels of one or more PBC associated antigens. In various embodiments, the particle surface is essentially free of one or more PBC associated antigens. In various embodiments, the amount of one or more PBC associated antigens present on the surface of the particles is between 0-30% (e.g., 0, 1, 2, 3, 4, 5, 6, 7, 8, 9, 10, 11, 12, 13, 14, 5, 16, 17, 18, 19, 20, 21, 22, 23, 24, 25, 26, 27, 28, 29, or 30% including all values and ranges that lie between these values) of the total antigen content of the TIMP-PBC composition. In various embodiments, the frequency of particles containing one or more PBC associated antigens on their surface is less than 30% or between 0-30% (e.g., 0, 1, 2, 3, 4, 5, 6, 7, 8, 9, 10, 11, 12, 13, 14, 5, 16, 17, 18, 19, 20, 21, 22, 23, 24, 25, 26, 27, 28, 29, or 30% including all values and ranges that lie between these values) higher compared to a negative control. In various embodiments, the frequency of particles containing one or more PBC associated antigens on their surface is 0-100% (e.g., 0, 1, 2, 3, 4, 5, 6, 7, 8, 9, 10, 20, 30, 40, 50, 60, 70, 80, 90, and 100% including all values and ranges that lie between these values) lower when compared to a positive control. In various embodiments, the amount of one or more PBC associated antigens on the surface of the particles is between 0-10-fold (e.g., 0, 1, 2, 3, 4, 5, 6, 7, 8, 9, or 10-fold including all values and ranges that lie between these values) higher than a negative control. In various embodiments, the amount of one or more PBC associated antigens on the surface of the particles is between 0-100-fold (e.g., 0, 1, 2, 3, 4, 5, 6, 7, 8, 9, 10, 20, 30, 40, 50, 60, 70, 80, 90, or 100-fold including all values and ranges that lie between these values) lower than a positive control. In various embodiments, the number of TIMP-PBC particles with one or more PBC associated antigens on their surface is determined using previously described methods such as flow cytometry, Mass Spectrometry, NMR, ELISA, CBQCA, and Western Blot.

In various embodiments, the present disclosure provides a process for manufacturing a composition comprising negatively charged particles encapsulating one or more PBC associated antigens (TIMP-PBC), wherein the particles exhibit low burst release. In various embodiments, the particles exhibit no burst release. In various embodiments, the particle burst release is between 0-75% (e.g., 0, 1, 2, 3, 4, 5, 6, 7, 8, 9, 10, 20, 30, 40, 50, 60, 70, or 75% including all values and ranges that lie between these values).

In various embodiments, excipients are added to the nanoparticle composition prior to freeze drying in step (f). In various embodiments, the excipients are buffering agents and/or cryoprotectants. In various embodiments, the excipients are selected from the group consisting of sucrose, mannitol, trehalose, sorbitol, dextran, Ficoll, Dextran 70k, sodium citrate, lactose, L-arginine, or glycine. In various embodiments, the amounts of excipients added to the nanoparticle composition prior to freeze drying is between 0.05 and 100% (e.g., between 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, or 100% including all values lying within this range) by weight or volume. In various embodiments, the amounts of excipients added to the nanoparticle composition prior to freeze drying is between 0.01 and 500 g (e.g., between 0.01, 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 150, 200, 250, 300, 350, 400, 450, or 500 g) per gram of nanoparticles.

In various embodiments, the manufacturing batch sizes of TIMP-PBC can be scaled up or down. In various embodiments, the manufacturing batch size is between 0.01 g to 100 kg. In various embodiments, the batch size is 0.01 g, 0.1 g, 10 g, 20 g, 40 g, 60 g, 80 g, 100 g, 160 g, 240 g, 320 g, 400 g, 480 g, 560 g, 640 g, 720 g, 800 g, 1000 g, 5 kg, 10 kg, 50 kg or 100 kg including all values and ranges that lie between these values.

Also provided is a composition comprising particles encapsulating PBC associated antigens made by the methods described herein. In various embodiments, the composition further comprises a pharmaceutically acceptable carrier, diluent, or excipient. In various embodiments, the pharmaceutical composition is a sterile pharmaceutical composition.

In various embodiments, formulations, or pharmaceutical compositions of TIMP-PBC contain negatively charged particles encapsulating PBC-associated antigens, and excipients. In various embodiments, the excipients are selected from the group consisting of sucrose, mannitol, trehalose, sorbitol, dextran, Ficoll, Dextran 70k, sodium citrate, lactose, L-arginine, or glycine. In various embodiments TIMP-PBC formulations contain between one to eleven excipients. In various embodiments, TIMP-PBC formulations contain one or more, two or more, three or more, four or more, five or more, six or more, seven or more, eight or more, nine or more, or ten or more excipients.

In various embodiments, TIMP-PBC formulations contain negatively charged particles encapsulating PBC associated antigens, sucrose, mannitol, and sodium citrate. In various embodiments, the negatively charged particle concentration in the TIMP-PBC formulation is between 1 to 100% including all ranges and values that lie between these ranges. In various embodiments, the negatively charged particle concentration in the TIMP-PBC formulation is between 20 to 50% including all ranges and values that lie between these ranges. In various embodiments, the negatively charged particle concentration in the TIMP-PBC formulation is between 30 to 40% including all ranges and values that lie between these ranges. In various embodiments, the negatively charged particle concentration in the TIMP-PBC formulation is about 30%, about 31%, about 32%, about 33%, about 34%, about 35%, about 35.6%, about 36%, about 37%, about 38%, about 39%, or about 40%.

In various embodiments, the sucrose concentration in the TIMP-PBC formulation is between 1 to 100% including all ranges and values that lie between these ranges. In various embodiments, the sucrose in the TIMP-PBC formulation is between 20 to 50% including all ranges and values that lie between these ranges. In various embodiments, the sucrose concentration in the TIMP-PBC formulation is between 30 to 40% including all ranges and values that lie between these ranges. In various embodiments, the sucrose concentration in the TIMP-PBC formulation is about 30%, about 31%, about 32%, about 33%, about 34%, about 35%, about 35.6%, about 36%, about 37%, about 38%, about 39%, or about 40%.

In various embodiments, the mannitol concentration in the TIMP-PBC formulation is between 1 to 100% including all ranges and values that lie between these ranges. In various embodiments, the mannitol concentration in the TIMP-PBC formulation is between 15 to 35% including all ranges and values that lie between these ranges. In various embodiments, the mannitol concentration in the TIMP-PBC formulation is between 20 to 30% including all ranges and values that lie between these ranges. In various embodiments, the mannitol concentration in the TIMP-PBC formulation is about 20%, about 21%, about 22%, about 23%, about 24%, about 25%, about 25%, about 26%, about 26.7%, about 27%, about 28%, about 29%, or about 30%.

In various embodiments, the sodium citrate concentration is between 0.01 to 25% including all ranges and values that lie between these ranges. In various embodiments, the sodium citrate concentration is between 0.5 to 3.5% including all ranges and values that lie between these ranges. In various embodiments, the sodium citrate concentration is about 0.5%, about 1%, about 1.5%, about 2%, about 2.1%, about 2.5%, about 3%, or about 3.5%.

The amount or concentration of PBC associated antigen in the TIMP-PBC formulation can be expressed as amount of PBC associated antigen (e.g., in μg) per mg of PLGA. In various embodiments, the amount of PBC-associated antigens in the TIMP-PBC formulation is between 0.1 μg/mg PLGA and 100 μg/mg PLGA including all ranges and values that lie between these ranges. In various embodiments, the amount of PBC-associated antigen in the TIMP-PBC formulation is between 0.1 μg/mg PLGA and 60 μg/mg PLGA, including all ranges and values that lie between these ranges. In various embodiments, the amount of PBC associated antigens in the TIMP-PBC formulation is between 0.5 μg/mg PLGA to 10 μg/mg PLGA, including all ranges and values that lie between these ranges. In some embodiments, the amount of PBC associated antigens in the TIMP-PBC formulation is between 1 μg/mg PLGA to 12 μg/mg PLGA including all ranges and values that lie between these ranges. In some embodiments, the amount of PBC associated antigens in the TIMP-PBC formulation is between 1 μg/mg PLGA to 5.8 μg/mg PLGA including all ranges and values that lie between these ranges. In various embodiments, the PBC associated antigens in the TIMP-PBC formulation is about 1 μg/mg PLGA, about 2 μg/mg PLGA, about 3 μg/mg PLGA, about 4 μg/mg PLGA, about 5 μg/mg PLGA, about 5.8 μg/mg PLGA, about 6 μg/mg PLGA, about 7 μg/mg PLGA, about 8 μg/mg PLGA, about 9 μg/mg PLGA, about 10 μg/mg PLGA, about 11 μg/mg PLGA, or about 12 μg/mg PLGA.

In various embodiments, the TIMP-PBC formulation comprises between about 10⁸ particles/mg PLGA and 10¹⁰ particles/mg PLGA including all ranges and values that lie between that range. In various embodiments, the TIMP-PBC formulation comprises about 10⁹ particles/mg PLGA.

In some embodiments, the amount of PBC-associated antigens encapsulated in a particle is between 0.01 femtograms/particle and 100 femtograms/particle, including all ranges and values that lie between these ranges. In various embodiments, the amount is between 0.01 femtograms/particle and 10 femtograms/particle, including all ranges and values that lie between these ranges. In various embodiments, the amount 0.05 femtograms/particle and 1.5 femtograms/particle, including all ranges and values that lie between these ranges. In some embodiments, the amount is between 0.1 femtograms/particle to 0.75 femtograms/particle, including all ranges and values that lie between these ranges. In various embodiments, the PBC associated antigens in the TIMP-PBC formulation is about 0.1 femtograms/particle, about 0.25 femtograms/particle, about 0.3 femtograms/particle, about 0.4 femtograms/particle, about 0.5 femtograms/particle, about 0.6 femtograms/particle, about 0.7 femtograms/particle, about 0.8 femtograms/particle, about 0.9 femtograms/particle, about 1 femtograms/particle, about 2 femtograms/particle, about 3 femtograms/particle, about 4 femtograms/particle, about 5 femtograms/particle, or about 6 femtograms/particle, including all ranges and values that lie between these ranges.

The disclosure provides for methods of treating PBC in a subject comprising administering to the subject particles encapsulating PBC associated antigens as described herein. Also contemplated is a composition comprising TIMP-PBC as described herein for use in treating PBC. In various embodiments, the disclosure provides for use of a composition comprising TIMP-PBC as described herein in the preparation of a medicament for treating PBC.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

### BRIEF DESCRIPTION OF THE FIGURES

FIG. 1. Exemplary manufacturing process flow diagram depicting steps involved in the manufacture of tolerizing nanoparticles encapsulating PBC associated antigens (TIMP-PBC).

FIG. 2. Physiochemical characterization of TIMP-PBC particles using Scanning Electron Microscopy (SEM). Shown is a representative SEM image of nanoparticles at 10,000× magnification.

### DETAILED DESCRIPTION

TIMPs are surface functionalized negatively charged particles made of biodegradable material encapsulating antigenic proteins or peptide epitopes associated with inflammatory conditions such as autoimmune diseases and allergies. TIMPs are designed for targeted delivery of encapsulated proteins/peptides to antigen presenting cells (APCs) of the mononuclear phagocyte system resulting in APC mediated T cell reprogramming via non-inflammatory pathways.

In pre-clinical models of autoimmune diseases and allergies, TIMPs have demonstrated therapeutic efficacy at inducing T-cell tolerance to antigenic/allergenic proteins and peptides resulting in improved disease symptoms.⁸⁻¹ TIMPs encapsulating one or more primary biliary cholangitis (PBC)-associated antigens (TIMP-PBC) can potentially treat PBC by reprogramming the immune system and inducing antigen specific T cell tolerance to PBC associated antigens. There is a current need for immune tolerizing therapies which can induce T-cell tolerance to autoimmune PBC associated antigens for long term therapeutic benefit without exposing patients to risk of adverse events.

The present disclosure provides a process for manufacturing negatively charged particles encapsulating one or more PBC associated antigens (TIMP-PBC) and pharmaceutical compositions comprising the particles.

### Definitions

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

“Particle” as used herein refers to any non-tissue derived composition of matter, it may be a sphere or sphere-like entity, bead, or liposome. The term “particle”, the term “immune modifying particle”, the term “carrier particle”, and the term “bead” may be used interchangeably depending on the context. Additionally, the term ‘particle’ may be used to encompass beads and spheres.

“Negatively charged particle” as used herein refers to particles which have been modified to possess a net surface charge that is less than zero.

“Surface-functionalized” as used herein refers to particles which have one or more functional groups on its surface. In some embodiments, the surface functionalization occurs by the introduction of one or more functional groups to a surface of a particle. In various embodiments, surface functionalization may be achieved by carboxylation (i.e., addition of one or more carboxyl groups to the particle surface) or addition of other chemical groups (e.g., other chemical groups that impart a negative surface charge).

“Carboxylated particles” or “carboxylated beads” or “carboxylated spheres” includes any particle that has been modified or surface functionalized to add one or more carboxyl group onto the particle surface. Carboxylation of the particles can be achieved using any compound which adds carboxyl groups, including, but not limited to, Poly (ethylene-maleic anhydride) (PEMA), Poly (acrylic acid), or a poly amino acid consisting of carboxyl side-chains (e.g., aspartic acid, glutamic acid). Carboxylation may also be achieved by using polymers with native carboxyl groups (e.g., PLGA) to form particles, in which the manufacturing process results in additional carboxyl groups, i.e., in addition to those naturally expressed by the polymer, being located on the surface of the particle.

As used herein the term “D90” refers to particle size distribution in which 90% of the total volume of material in the sample is contained or found. For example, if the D90 is 1000 nm, this indicates that 90% of the sample has a size of 1000 nm or smaller. Similarly, D50 or D10 refers to a particle size distribution in which 50% or 10%, respectively, of the total volume of material in the sample is contained or found. D [4,3] refers to volume or mass moment mean or Dc Brouckere Mean Diameter. Particle size is measured using techniques known in the art. In some embodiments, the D90/D50/D10 are number (count or frequency) based measurements or volume-based measurements which can be carried out by Laser Diffraction (LD), SPOS, or SPOS with nanoparticle tracking analysis (SPOS-NTA).

“Polypeptide” and “protein” refer to a polymer composed of amino acid residues, related naturally occurring structural variants, and synthetic non-naturally occurring analogs thereof, linked via peptide bonds or peptide bond isosteres. Synthetic polypeptides can be synthesized, for example, using an automated polypeptide synthesizer. The terms “polypeptide” and “protein” are not limited to a minimum length of the product. The term “protein” typically refers to large polypeptides. The term “peptide” typically refers to short polypeptides. Thus, peptides, oligopeptides, dimers, multimers, and the like, are included within the definition. Both full-length proteins and fragments thereof are encompassed by the definition. The terms “polypeptide” and “protein” also include post-expression modifications of the polypeptide or protein, for example, glycosylation, acetylation, phosphorylation and the like. Furthermore, for purposes of the present disclosure, a “polypeptide” can include “modifications,” such as deletions, additions, substitutions (which may be conservative in nature or may include substitutions with any of the 20 amino acids that are commonly present in human proteins, or any other naturally or non-naturally occurring or atypical amino acids), and chemical modifications (e.g., addition of or substitution with peptidomimetics), to the native sequence. These modifications may be deliberate, as through site-directed mutagenesis, or through chemical modification of amino acids to remove or attach chemical moieties, or may be accidental, such as through mutations arising via hosts cells that produce the proteins or through errors due to PCR amplification prior to host cell transfection.

“Antigenic moiety” or “antigen” as used herein refers to any moiety, for example a peptide, that is recognized by the host's immune system. Examples of antigenic moieties include, but are not limited to, autoantigens, allergens, enzymes, and/or bacterial or viral proteins, peptides, drugs or components.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration) or via inhalation.

The term “pharmaceutically acceptable” or “pharmacologically acceptable” refers to material that is not biologically or otherwise undesirable, i.e., the material may be administered to an individual without causing any undesirable biological effects or without interacting in a deleterious manner with any of the components of the composition in which it is contained or with any components present on or in the body of the individual.

The term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous. Moreover, epitopes may be mimetic (mimotopes) in that they comprise a three-dimensional structure that is identical to the epitope used to generate the antibody yet comprise none or only some of the amino acid residues found in the target that were used to stimulate the antibody immune response. As used herein, a mimotope is not considered a different antigen from the epitope bound by the selective binding agent; the selective binding agent recognizes the same three-dimensional structure of the epitope and mimotopc. “Epitope” as used herein is also known as an “antigenic determinant”, which is the part of an antigen that is recognized by the immune system, specifically by antibodies, B cells, or T cells. For example, an epitope is a specific piece of the antigen that an antibody binds to. The part of an antibody that binds to the epitope is called a paratope. Although epitopes are usually non-self-proteins, sequences derived from the host that can be recognized (as in the case of autoimmune diseases) are also epitopes. T cell epitopes are presented on the surface of an antigen-presenting cell, where they are bound to MHC (major histocompatibility complex) molecules. In humans, professional antigen-presenting cells are specialized to present MHC class II peptides, whereas most nucleated somatic cells present MHC class I peptides. T cell epitopes presented by MHC class I molecules are typically peptides between 8 and 11 amino acids in length, whereas MHC class II molecules present longer peptides, 13-17 amino acids in length, and non-classical MHC molecules also present non-peptidic epitopes such as glycolipids

The terms “treat”, “treated”, “treating” and “treatment”, as used with respect to methods herein refer to eliminating, reducing, suppressing or ameliorating, either temporarily or permanently, either partially or completely, one or more clinical symptom, manifestation or progression of an event, disease or condition. Such treating need not be absolute to be useful. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### Particles

The size and charge of the particles are important for tolerance induction. While the particles will differ in size and charge based on the antigen encapsulated within them, in general, particles described herein are effective at inducing tolerance when they are between about 100 nanometers and about 1500 nanometers and have a negative charge of between 0 to about −100 mV. In various embodiments, the particles are 400-800 nanometers in diameter and have a charge of between about −25 mV and −70 mV. In various embodiments, the particles are 400-1000 nanometers in diameter and have a charge of between about −25 mV and −70 mV. The average particle size and charge of the particles can be slightly altered in the lyophilization process, therefore, both post-synthesis averages and post-lyophilization averages are described. As used herein, the term “post-synthesis size” and “post synthesis charge” refer to the size and charge of the particle prior to lyophilization. The term “post lyophilization size” and “post lyophilization charge” refer to the size and charge of the particle after lyophilization.

In some embodiments, the particle is non-metallic. In these embodiments the particle may be formed from a polymer. In a preferred embodiment, the particle is biodegradable in an individual. In this embodiment, the particles can be provided in an individual across multiple doses without there being an accumulation of particles in the individual. Examples of suitable particles include polystyrene particles, PGA particles, PLA particles, PLGA particles, PLURONICS stabilized polypropylene sulfide particles, polypropylene sulfone, poly(ethylene glycol)-block-poly(propylene sulfide) copolymer, liposomes and diamond particles.

In some embodiments, the particle surface is composed of a material that minimizes non-specific or unwanted biological interactions. Interactions between the particle surface and the interstitium may be a factor that plays a role in lymphatic uptake. The particle surface may be coated with a material to prevent or decrease non-specific interactions. Steric stabilization by coating particles with hydrophilic layers such as poly (ethylene glycol) (PEG) and its copolymers such as PLURONICS® (including copolymers of poly (ethylene glycol)-bl-poly (propylene glycol)-bl-poly (ethylene glycol)) may reduce the non-specific interactions with proteins of the interstitium as demonstrated by improved lymphatic uptake following subcutaneous injections. All of these facts suggest relevance of the physical properties of the particles in terms of lymphatic uptake. Biodegradable polymers may be used to make all or some of the polymers and/or particles and/or layers. Biodegradable polymers may undergo degradation, for example, by a result of functional groups reacting with the water in the solution. The term “degradation” as used herein refers to becoming soluble, either by reduction of molecular weight or by conversion of hydrophobic groups to hydrophilic groups. Polymers with ester groups are generally subject to spontaneous hydrolysis, e.g., polylactides and polyglycolides.

Particles disclosed herein may also contain additional components. For example, carriers may have imaging agents incorporated or conjugated to the carrier. An example of a carrier nanosphere having an imaging agent that is currently commercially available is the Kodak X-sight nanospheres. Inorganic quantum-confined luminescent nanocrystals, known as quantum dots (QDs), have emerged as ideal donors in FRET applications: their high quantum yield and tunable size-dependent Stokes Shifts permit different sizes to emit from blue to infrared when excited at a single ultraviolet wavelength. (Bruchez, et al., Science, 1998, 281, 2013; Niemeyer, C. M Angew. Chem. Int. Ed. 2003, 42, 5796; Waggoner, A. Methods Enzymol. 1995, 246, 362; Brus, L. E. J. Chem. Phys. 1993, 79, 5566). Quantum dots, such as hybrid organic/inorganic quantum dots based on a class of polymers known as dendrimers, may be used in biological labeling, imaging, and optical biosensing systems. (Lemon, et al., J. Am. Chem. Soc. 2000, 122, 12886). Unlike the traditional synthesis of inorganic quantum dots, the synthesis of these hybrid quantum dot nanoparticles does not require high temperatures or highly toxic, unstable reagents. (Etienne, et al., Appl. Phys. Lett. 87, 181913, 2005).

Particles can be formed from a wide range of materials. The particle is preferably composed of a material suitable for biological use. For example, particles may be composed of glass, silica, citrate, polyesters of hydroxy carboxylic acids, polyanhydrides of dicarboxylic acids, or copolymers of hydroxy carboxylic acids and dicarboxylic acids. More generally, the carrier particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, carrier particles can be quantum dots, or composed of quantum dots, such as quantum dot polystyrene particles (Joumaa et al. (2006) Langmuir 22:1810-6). Carrier particles including mixtures of ester and anhydride bonds (e.g., copolymers of glycolic and sebacic acid) may also be employed. For example, carrier particles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), poly(lactic-co-sebacic) acid copolymers (PLSA), poly(glycolic-co-sebacic) acid copolymers (PGSA), polypropylene sulfide polymers, polypropylene sulfone, poly (ethylene glycol)-block-poly (propylene sulfide) copolymer, poly(caprolactone), chitosan, etc. Other biocompatible, biodegradable polymers useful in the present invention include polymers or copolymers of caprolactones, carbonates, amides, amino acids, orthoesters, acetals, cyanoacrylates, and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include diamond, PLA, PGA, polypropylene sulfide, and PLGA polymers. Biocompatible but non-biodegradable materials may also be used in the carrier particles of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

In certain embodiments, the particle is a co-polymer having a molar ratio from about 80:20 to about 100:0, or about 20:80 to 100:0. Suitable co-polymer ratio of present immune modified particles may be 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0. In certain embodiments, the particle is a PLURONICS stabilized polypropylene sulfide particle, a polyglycolic acid particle (PGA), a polylactic acid particle (PLA), a poly (lactic-co-glycolic acid) particle, a carboxylated polyglycolic acid particle (PGA), carboxylated polylactic acid particle (PLA), or carboxylated poly (lactic-co-glycolic acid) particle. In certain embodiments, the particle has a copolymer ratio of polylactic acid/polyglycolic acid 80:20:polylactic acid/polyglycolic acid 90:10:or polylactic acid/polyglycolic acid 50:50. In various embodiments, the particle is a poly (lactic-co-glycolic acid) particle and has a copolymer ratio of about 50:50 polylactic acid:polyglycolic acid.

It is contemplated that the particle may further comprise a surfactant and/or stabilizer. The surfactant and/or stabilizer can be anionic, cationic, or nonionic. Surfactants in the poloxamer and polaxamine family are commonly used in particle synthesis. Surfactants and/or stabilizers that may be used, include, but are not limited to PEG, Tween-80, gelatin, dextran, Pluronic L-63, PVA, PAA, methylcellulose, lecithin, DMAB and PEMA. Additionally, biodegradable and biocompatible surfactants including, but not limited to, vitamin E TPGS (D-a-tocopheryl polyethylene glycol 1000 succinate), poly amino acids (e.g., polymers of lysine, arginine, aspartic acid, glutamic acid, serine, threonine, tyrosine and cysteine, or their enantiomers), sodium cholate, and sulfate polymers. In certain embodiments, two surfactants are used. In certain embodiments, two stabilizers are used. In certain embodiments, a combination of two or more surfactants and stabilizers are used. For example, if the particle is produced by a double emulsion method, the two surfactants can include a hydrophobic surfactant for the first emulsion, and a hydrophobic surfactant for the second emulsion. For example, stabilizers can be compounds which stabilize the primary and/or the secondary emulsion as described herein by providing a physical barrier or an energy barrier between adjacent nanoparticle droplets in the emulsion, thereby reducing their probability to coalesce and form larger nanoparticle droplets.

In various embodiments, the polypeptide antigens are encapsulated in the particles by a single-emulsion process. In a further embodiment, the polypeptide antigens are more hydrophobic. Sometimes, the double emulsion process leads to the formation of large particles which may result in the leakage of the hydrophilic active component and low entrapment efficiencies. The coalescence and Ostwald ripening are two mechanisms that may destabilize the double-emulsion droplet, and the diffusion through the organic phase of the hydrophilic active component is the main mechanism responsible of low levels of entrapped active component. In some embodiments, it may be beneficial to reduce the nanoparticle size. One strategy to accomplish this is to apply a second strong shear rate. The leakage effect can be reduced by using a high polymer concentration and a high polymer molecular mass, accompanied by an increase in the viscosity of the inner water phase and in increase in the surfactant molecular mass. In certain embodiments, the particles encapsulating antigens are manufactured by nanoprecipitation, co-precipitation, inert gas condensation, sputtering, microemulsion, sol-gel method, layer-by-layer technique or ionic gelation method.

TIMP-PBC particles can be synthesized by a nanoprecipitation method with or without the assistance of a microfluidic device. In the nanoprecipitation method without the use of a microfluidic device, a biodegradable polymer (e.g. PLA, PGA, PLGA) is dissolved in one or more organic solvents, or a combination thereof. In some embodiments, the organic solvent is selected from the group comprising acetone, acctonitrile, dichloromethane, chloroform, tetrahydrofuran, ethyl acetate, ethyl formate, or dimethyl carbonate. The polymer is dissolved at concentrations of about 1.0 mg/mL and 100 mg/mL including all ranges and values lying within that range. The polymer solution is added to an aqueous solution containing the PBC associated antigen. In some embodiments, the PBC associated antigen is PDC-E2. In some embodiments the addition is performed while stirring the mixture. In some embodiments the stirring is magnetic. The mixture is then added to another mixture of higher volume including one or more surfactants. In some embodiments, the surfactant is selected from the group comprising poloxamer, a polyamine, PEG, Tween-80, gelatin, dextran, pluronic L-63, pluronic F-68, pluronic 188, pluronic F-127, PVA, PAA, methylcellulose, lecithin, DMAB, PEMA, vitamin E TPGS (D-a-tocopheryl polyethylene glycol 1000 succinate), hyaluronic acid, poly amino acids, poly lysine, poly arginine, poly aspartic acid, poly glutamic acid, polyserine, polythreonine, polytyrosine, poly cysteine, enantiomers of poly amino acids, sodium cholate, methylcellulose, hydroxyethylcellulose, hydroxyprolylcellulose, hydroxypropylmethylcellulose, gelatin, a carbomer, and a sulfate polymer. The organic solvent can be removed by solvent diffusion, or solvent evaporation. In some embodiments, the evaporation is active evaporation. In some embodiments, the evaporation is active evaporation under vacuum followed by passive overnight evaporation. The particles are washed by TFF. TIMP-PBC particles can be synthesized by a nanoprecipitation method using one or more microfluidic devices. In some embodiments, the devices are selected from the group comprising continuous microfluidic mixing, Y- and T-shaped microfluidic devices, hydrodynamic Flow focusing-HFF, Impinging jets mixer and multi-inlet vortex mixer. Monodisperse particles of size 400-800 nm can be obtained with a low PDI. The method involves adjusting the total flow rate (TFR) and the flow rate ratios (FRR) of three solutions:
(i) Biodegradable polymer containing organic phase. In some embodiments, the polymers is selected form the group comprising PLA, PGA, PLGA,
(ii) The PBC associated antigen containing aqueous phase 1 (W₁). In some embodiments, the PBC associated antigen is PDC-E2 peptide.
(iii) The surfactant containing phase W₂. In some embodiments, the surfactant is selected from the group comprising poloxamer, a polyaminc, PEG, Tween-80, gelatin, dextran, pluronic L-63, pluronic F-68, pluronic 188, pluronic F-127, PVA, PAA, methylcellulose, lecithin, DMAB, PEMA, vitamin E TPGS (D-a-tocopheryl polyethylene glycol 1000 succinate), hyaluronic acid, poly amino acids, poly lysine, poly arginine, poly aspartic acid, poly glutamic acid, polyserine, polythreonine, polytyrosine, poly cysteine, enantiomers of poly amino acids, sodium cholate, methylcellulose, hydroxyethylcellulose, hydroxyprolylcellulose, hydroxypropylmethylcellulose, gelatin, a carbomer, and a sulfate polymer.

The organic solvent can be removed by solvent diffusion or solvent evaporation. In some embodiments, the evaporation is active evaporation. In some embodiments, the evaporation is active evaporation under vacuum followed by passive overnight evaporation. The particles are washed by TFF.

Several methods for manufacturing nanoparticles have been described in the literature and are incorporated herein by reference^{3,4}.

### Antigens

An antigen refers to a discreet portion of a molecule, such as a polypeptide or peptide sequence, a 3-D structural formation of a polypeptide or peptide, a polysaccharide or polynucleotide that can be recognized by a host immune cell. Antigen-specific refers to the ability of a subject's host cells to recognize and generate an immune response against an antigen alone, or to molecules that closely resemble the antigen, as with an epitope or mimotope.

“Anergy,” “tolerance,” or “antigen-specific tolerance” refers to insensitivity or reprogramming of T cells to T cell receptor-mediated stimulation. Such reprogramming is generally antigen-specific and persists after exposure to the antigenic peptide has ceased. This reprogramming leads to induction of regulatory T cells (Treg), Tr1 cells and T cell anergy. For example, insensitivity in T cells is characterized by lack of effector cytokine production, lack of proliferation, or lack of activation. Reprogramming occurs when T cells are exposed to antigen and receive a first signal (a T cell receptor or CD3 mediated signal) in the absence of a second signal (a costimulatory signal), or presence of an inhibitory signal (negative costimulation or regulatory cytokines). Under these conditions, re-exposure of the cells to the same antigen (even if re-exposure occurs in the presence of a costimulatory molecule) results in failure to produce cytokines and subsequently failure to proliferate. Thus, a failure to produce cytokines prevents proliferation. Anergic T cells can, however, proliferate if cultured with certain agents (e.g., IL-2).

It is contemplated that the tolerizing therapy described herein is antigen-specific. For example, TIMPs administered as tolerizing therapy encapsulate one or more antigens associated with said tolerizing therapy and associated disease or condition being treated. It is contemplated that the TIMPs used in tolerizing therapy comprise one or more PBC associated antigens.

In various embodiments, TIMP-PBC encapsulates one or more PBC associated antigens or antigenic epitopes thereof. In various embodiments, the PBC associated antigens are intracellular proteins, extracellular proteins, mitochondrial proteins, and/or nuclear proteins. In various embodiments, the TIMP-PBC particles encapsulate one or more polynucleotides encoding PBC associated antigens. In some embodiments, the polynucleotides comprise DNA, RNA, messenger RNA (mRNA), or circular RNA.

In various embodiments, the PBC associated antigens are selected from the group consisting pyruvate dehydrogenase complex E2 subunit (dihydrolipoyllysine-residue acetyltransferase component of pyruvate dehydrogenase complex) (PDC-E2), gp210, nucleoporin 62, Sp100, PML, CENP A, CENP B, CENP C, dsDNA, histone, branched-chain 2-oxo-acid dehydrogenase complex (BCOADC), lipoamide acyltransferase component of branched-chain alpha-keto acid dehydrogenase complex (BCOADC-E2), dihydrolipoyllysine-residue succinyltransferase component of 2-oxoglutarate dehydrogenase complex (OGDC-E2), sulfite oxidase, outer mitochondrial membrane, glycogen phosphorylase, sarcosine dehydrogenase, smooth muscle protein, soluble liver antigen, liver/kidney microsome, centromere protein, tubulin, actin, vimentin, desmin, cytokeratin, F-actin, UDP glucuronosyltransferase Family 1 Member A Complex (UGTA1), formiminotransferase cyclodeaminase, asialoglycoprotein receptor (ASGPR), cardiolipin, h-Lamp-2, proteinase 3, CYP 2C9, CYP 2A6, and CYP P450 2D6. In some embodiments, the PBC-associated antigens are selected from the group comprising bacterial epitopes, viral epitopes, or xenobiotics. In In some embodiments the bacterial epitopes, viral epitopes, or xenobiotics comprise 2-octynamide, 2-nonynamide, Escherichia coli PDC-E2, Escherichia coli ATP dependent ClpX, Escherichia coli periplasmic maltose-binding protein, Escherichia coli ATP dependent helicase Hrp, Escherichia coli fatty acid oxidation complex alpha, Escherichia coli ppGpp synthetase II, Escherichia coli nitrate reductase 2, Helicobacter pylori urcasc beta subunit, Pseudomonas aeruginosa diaminopimelate decarboxylase, human cytomegalovirus capsid assembly protein UL47, Haemophilus influenzae t-RNA (uracil-5-)-methyltransferase, Novosphingobium aromaticivorans proteins Novo 1, Novo 2, Novo 3, Novo 4, or Lactobacillus delbrueckii beta-galactosidase.

In various embodiments, TIMP-PBC encapsulates one or more PDC-E2 peptides comprising an antigenic epitope. In various embodiments, TIMP-PBC encapsulates an antigenic epitope comprising PDC-E2₁₅₅₋₁₈₅. In various embodiments, TIMP-PBC encapsulates PDC-E2 antigenic epitope amino acids 155-185 (PDC-E2₁₅₅₋₁₈₅), having amino acid sequence KVGEKLSEGDLLAEIETDKATIGFEVQEEGY) (SEQ ID NO: 1).

In certain embodiments, one, two, three, or a higher number of antigens or antigenic peptides are used in the TIMPs. In certain embodiments, the one or more PBC associated antigens are encapsulated in the TIMP by covalent linkage to the interior surface of the particle (See e.g., US Patent Publication US20190282707, herein incorporated by reference). In certain embodiments, it is contemplated that sequences of two or more PBC associated antigens are linked in a fusion protein and encapsulated within a TIMP described herein. Methods for making TIMP with linked epitopes are described in US Patent Publication US20190365656, herein incorporated by reference.

In certain embodiments, chemicals, residuals, or other impurities are removed or reduced from the antigens or antigenic peptides prior to encapsulation in the particles. In certain embodiments, chemicals, residuals, or other impurities are removed or reduced from the antigens or antigenic peptides prior to or during generation of the primary emulsion. In certain embodiments the residual is trifluoroacetic acid (TFA). Residuals may be removed or reduced by reverse osmosis (dialysis), lyophilization, ultrafiltration, washing, HPLC, reverse-phase HPLC, ion-exchange resin, washing with trifluoroethanol or any other method known in the art.

Emulsions occur in many forms of processing and are used extensively by the food, cosmetics and drug delivery. Oil-water (single), water-oil-water (double) emulsion, and/or solid-oil-water emulsion are methods by which PLGA or other biodegradable polymers can be used to encapsulate hydrophobic and hydrophilic drugs in micro- or nanoscale form. For example, PLGA is dissolved into an organic phase (oil) that is emulsified with a surfactant or stabilizer (water). “Oil phase” refers to an organic solvent immiscible in water. Hydrophobic drugs and/or other agents are added directly to the oil phase, whereas hydrophilic drugs and/or other agents (water) may be first emulsified with the polymer solution prior to formation of particles. High intensity homogenization (e.g., sonication bursts) or other high energy input facilitate the formation of small polymer droplets. The resulting emulsion is added to a larger aqueous phase and stirred for several hours, which allows the solvent to evaporate. Hardened nanoparticles are collected and washed by centrifugation. In certain embodiments, hardened emulsion particles can be obtained through evaporation of the oil phase.

“Water-in-oil-in-water” (W/O/W) emulsion is an example of a double emulsion, in which dispersions of small water droplets within larger oil droplets are themselves dispersed in a continuous aqueous phase. Because of their compartmentalized internal structure, double emulsions can provide advantages over simple oil-in-water emulsions for encapsulation, such as the ability to carry both polar and non-polar cargos (pharmaceutical/biological agent, e.g., proteins), and improved control over release of therapeutic molecules. The preparation of double emulsions typically requires surfactants or their mixtures for stability. The surfactants stabilize droplets subjected to extreme flow, leading to direct, mass production of robust double nanoemulsions that are amenable to nanostructured encapsulation applications in various industries. In one example, a double emulsion process involves generating a primary emulsion by mixing an aqueous solution of a pharmaceutical/biological agent(s) with a solution including a polymer resulting in a water-in-oil primary emulsion. The primary emulsion is then mixed with a solution including one or more surfactants to form an oil-in-water secondary emulsion. The secondary emulsion is then hardened by evaporation to remove the solvent(s) resulting in hardened polymeric nanoparticles encapsulating the pharmaceutical/biological agent(s).

“Homogenization” as used herein relates to an operation using a class of processing equipment referred to as homogenizers that are geared towards reducing the size of droplets in liquid-liquid dispersions. Factors that affect the particle or droplet size include but are not limited to the type of emulsifier, emulsifier concentration, solution conditions, and mechanical device (homogenizing power; pressure, rotation speed, time). Non-limiting examples of homogenizers include high speed blender, high pressure homogenizers, colloid mill, high shear dispersers, ultrasonic disruptor membrane homogenizers, and ultrasonicators. Mechanical homogenizers, manual homogenizers, sonicators, mixer mills, vortexers, and the like may be utilized for mechanical and physical disruption within the scope of the disclosure.

“Batch size” as used herein relates to the scale of manufacture depending on the weight of the particles in the final product. The manufacturing process can be altered, scaled up or scaled down. The manufacturing process can be altered, scaled up or scaled down by altering the amount or volume of the solvent, antigens/proteins, polymer, surfactants, stabilizers, cryoprotectants or excipients. The manufacturing process can be scaled up or down by altering the time of homogenization, sonication, evaporation, filtration, concentration, washing or lyophilization. Number of vials filled with TIMP-PBC will vary depending on batch size. About 1, 5, 50, 500, 5000, 50,000 or 500,000 vials are filled including all values and ranges that lie in between these values. Amount of TIMP-PBC filled in each vial is about 0.01 mg, about 0.1 mg, about 1 mg, about 10 mg, about 100 mg, about 200 mg, or about 250 mg including all values and ranges that lie in between these values.

Methods for determining protein content in the particles or in solution include ELISA, Mass Spectrometry, HPLC, CBQCA, and Western Blot.

Molecular Probes CBQCA Protein Quantitation Kit provides a rapid and highly sensitive method for the quantitation of proteins in solution. The kit utilizes the ATTO-TAG CBQCA reagent (3-(4-carboxybenzoyl) quinoline-2-carboxaldehyde) originally developed as a chromatographic derivatization reagent for amines. This reagent has also proven extremely useful for quantitating amines in solution, including the accessible amines in proteins. The ATTO-TAG CBQCA reagent is virtually non-fluorescent in aqueous solution; however, in the presence of cyanide, it reacts with primary amines such as those found in proteins to form highly fluorescent derivatives.

### Acid

An acid is a molecule or ion capable of either donating a proton (i.e., hydrogen ion, H⁺), or forming a covalent bond with an electron pair.

An aqueous solution of an acid has a pH less than 7. A lower pH means a higher acidity, and thus a higher concentration of positive hydrogen ions in the solution. Chemicals or substances having the property of an acid are said to be acidic.

In various embodiments the acid is acetic acid, sulfuric acid, hydrochloric acid, nitric acid, formic acid, benzoic acid, ascorbic acid, trichloroacetic acid, dichloroacetic acid, chloroacetic acid, trifluoroacetic acid, fluoroacetic acid, tartaric acid, lactic acid, gluconic acid, methanesulfonic acid, ethanesulfonic acid, benzenesulfonic acid, p-toluenesulfonic acid, trifluoromethanesulfonic acid, polystyrene sulfonic acid, hydrobromic, hydroiodic acid, hypochlorous acid, chloric acid, chloric acid, perchloric acid, fluorosulfuric acid, fluoroantimonic acid, fluoroboric acid, hexafluorophosphoric acid, chromic acid, phosphoric acid, hydrofluoric acid, oxalic acid, boric acid, carbonic acid.

### Base

A base is a chemical species that donates electrons, accepts protons, or releases hydroxide (OH−) ions in aqueous solution.

An aqueous solution of a base has a pH more than 7.0 and up to 14.0. A higher pH means a higher basicity, and thus a higher concentration of negative hydroxide ions in the solution. Chemicals or substances having the property of a base are said to be basic.

In various embodiments, the base is barium hydroxide, calcium hydroxide, chromium hydroxide, potassium hydroxide, ammonium hydroxide, zinc hydroxide, barium hydroxide, sodium bicarbonate, methylamine, diethylamine, sodium hydroxide, magnesium hydroxide, ammonium bicarbonate, ammonia aluminium hydroxide, sodium carbonate, magnesium hydroxide, zinc hydroxide, ferrous hydroxide, acetone, lithium hydroxide, pyridine, rubidium hydroxide.

### Pharmaceutical Formulations

Pharmaceutical compositions of the present disclosure containing the TIMP-PBC described herein as an active ingredient may contain pharmaceutically acceptable carriers or additives depending on the route of administration. Examples of such carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present disclosure.

Formulation of the pharmaceutical composition will vary according to the route of administration selected (e.g., solution, emulsion). An appropriate composition comprising the therapeutic to be administered can be prepared in a physiologically acceptable vehicle or carrier. For solutions or emulsions, suitable carriers include, for example, aqueous or alcohol-ic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the particles are prepared for storage by mixing the particle having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl para-bens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn— protein complexes); and/or non-ionic surfactants such as TWEEN™, PLURONICS™ or polyethylene glycol (PEG).

The formulations to be used for in vivo administration must be sterile. This is readily accomplished by filtration through sterile filtration membranes.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The TIMP-PBC described herein can be lyophilized for storage and reconstituted in a suitable carrier prior to use.

Solid dosage forms for oral administration include capsules, tablets, pills, powders, and granules. In such solid dosage forms, the modified particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

### Methods of Use

Provided herein is a method of treating primary biliary cholangitis (PBC) in a subject comprising administering to the subject TIMP-PBC described herein, wherein TIMP-PBC is administered at a dose of 0.01 to 12 mg/kg. Also provided herein is a method of reducing an inflammatory immune response to PBC antigens in a subject suffering from PBC comprising administering to the subject TIMP-PBC, wherein TIMP-PBC is administered at a dose of 0.01 to 12 mg/kg.

Provided herein is a method of treating PBC in a subject comprising administering to the subject TIMP-PBC, wherein TIMP-PBC is administered at a dose determined based on the subject's weight. In various embodiments, TIMP-PBC is administered at a dose of 0.01 to 12 mg/kg. In various embodiments, TIMP-PBC is administered at a fixed dose of between 1 mg to 800 mg. In various embodiments, the TIMP-PBC is administered at a dose from about from about 0.01 to about 12 mg/kg, from about 0.05 to about 10 mg/kg, from about 0.01 to about 5 mg/kg, from about 0.1 to about 10 mg/kg, from about 1 to about 8 mg/kg, from about 1.5 to about 10 mg/kg, from about 2 to about 12 mg/kg, from about 2 to about 10 mg/kg, from about 3 to about 10 mg/kg, from about 4 to about 10 mg/kg, from about 4 to 12 mg/kg, or from about 5 to about 12 mg/kg. In various embodiments, TIMP-PBC is administered at a dose of about 0.01 mg/kg, about 0.05 mg/kg, about 0.1 mg/kg, about 0.25 mg/kg, about 0.5 mg/kg, about 1.0 mg/kg, about 2.0 mg/kg, about 3.0 mg/kg, about 4.0 mg/kg, about 5 mg/kg, about 6 mg/kg, about 8.0 mg/kg, about 10 mg/kg, or about 12 mg/kg. In various embodiments, TIMP-PBC is administered at a fixed dose of about 1 mg, about 2 mg, about 5 mg, about 10 mg, about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, or about 800 mg.

Also contemplated, the TIMP-PBC is administered at a concentration of between about 0.005 mg/mL and about 50 mg/mL. In various embodiments, TIMP-PBC is administered at a concentration of about 0.05 mg/mL, 0.1 mg/mL, 0.5 mg/mL, 1 mg/mL, 2 mg/mL, 3 mg/mL, 3.25 mg/ml, 3.5 mg/ml, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL. In various embodiments, TIMP-PBC is administered via intravenous infusion lasting about 1, 2, 3, 4, 5, 6, 7, or 8 hours.

It is contemplated that the TIMP-PBC is administered in a single dose or in multiple doses. In various embodiments, TIMP-PBC is administered once weekly, once every two weeks, once every three weeks, once every 4 weeks, once every two months, once every three months, once every 6 months, once per year, once every two years, once every three years, once every four years, once every five years, once every six years, once every seven years, once every eight years, once every nine years, or once every ten years. In certain embodiments, TIMP-PBC is administered in two doses one-week apart.

In various embodiments, TIMP-PBC is administered intravenously, subcutaneously, intramuscularly, intraperitoneally, intranasally, inhaled or orally. It is contemplated that if TIMP-PBC is given intravenously, it can be via intravenous infusion lasting about 1, 2, 3, 4, 5, 6, 7, or 8 hours.

TIMP-PBC therapy is contemplated to relieve, lessen or ameliorate one or more symptoms of primary biliary cholangitis (PBC). Symptoms of PBC include, but are not limited to, liver inflammation, cirrhosis, cholestasis, liver dysfunction, liver failure, liver fibrosis, increased liver immune infiltrate, elevated bile acid levels, elevated liver enzyme levels, (ALT, ALP, AST, y-glutamyl transpeptidase), elevated bilirubin levels, circulating anti-mitochondrial antibodies (AMAs), circulating anti-nuclear antibodies (ANAs), fatigue, itchy skin, pruritis, dry eyes and mouth, abdominal pain, splenomegaly, musculoskeletal pain, edema, fluid buildup, skin xanthomas, jaundice, hyperpigmentation, osteoporosis, high cholesterol, diarrhea, steatorrhea, hypothyroidism, and weight loss. Diagnosis and symptoms of PBC are measured using methods known in the art, such as analysis of liver pathology by histology, fibroscan and/or MR elastography.

TIMP-PBC therapy is also contemplated to reduce, shorten or ameliorate the duration and severity of an inflammatory immune response to one or more PBC antigens in a subject. An inflammatory immune response includes a T cell response, B cell response, Th1 response, myeloid cell response, and/or an antibody response. In various embodiments, the efficacy of TIMP-PBC at relieving one or more symptoms of PBC and/or reducing the duration and severity of an inflammatory immune response to one or more PBC antigens is determined from the assay of one or more biological samples from the subject as described herein.

Also provided herein is a method of treating autoimmune diseases where PDC-E2 is implicated as an antigen. In some embodiments, the method comprises administering tolerizing nanoparticles encapsulating PDC-E2 antigens for treating PDC-E2 associated autoimmune diseases. In various embodiments, the PDC-E2 associated autoimmune disorders are selected from the group comprising PBC, Sjogren's syndrome, scleroderma, systemic lupus erythematosus, rheumatoid arthritis, or autoimmune hepatitis. The disclosure provides for methods of treating PDC-E2 associated autoimmune disease in a subject comprising administering to the subject particles encapsulating PDC-E2 as described herein. Also contemplated is a composition comprising TIMPs encapsulating PDC-E2 as described herein for use in treating PBC, Sjogren's syndrome, scleroderma, systemic lupus erythematosus, rheumatoid arthritis, or autoimmune hepatitis. In various embodiments, the disclosure provides for use of a composition comprising TIMPs encapsulating PDC-E2 as described herein in the preparation of a medicament for treating PBC, Sjogren's syndrome, scleroderma, systemic lupus erythematosus, rheumatoid arthritis, or autoimmune hepatitis.

### Kits

As an additional aspect, the disclosure includes kits which comprise one or more compounds or compositions packaged in a manner which facilitates their use to practice methods of the disclosure. In one embodiment, such a kit includes a compound or composition described herein (e.g., a composition comprising a TIMP alone or in combination with a second agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the inhibitor compositions.

In a further embodiment, the disclosure provides an article of manufacture, or unit dose form, comprising: (a) a composition of matter comprising TIMP-PBC as described herein; (b) a container containing said composition; and (c) a label affixed to said container, or a package insert included in said container referring to the use of said TIMP-PBC in the treatment of PBC as described herein.

Additional aspects and details of the disclosure will be apparent from the following examples, which are intended to be illustrative rather than limiting.

### EXAMPLES

### Example 1. Process for Preparing PDC-E2 Peptide Solution for the Manufacture of Tolerizing Nanoparticles Encapsulating PBC Associated Antigens (TIMP-PBC)

To prepare the antigen solution, PDC-E2 peptide is added to 0.1% ammonium hydroxide to an interim concentration of 14 mg/mL PDC-E2 peptide. The solution is stirred to ensure that the peptide is completely dissolved. Next, the PDC-E2 peptide solution is pH adjusted using the 1N acetic acid to a pH of between 1.0 to 7.0. The adjusted solution is then further diluted to the final 8.25 mg/mL PDC-E2 peptide concentration by adding water for injection (WFI) and mixed until homogenous.

In an alternate method, PDC-E2 peptide is added to 3% ammonium hydroxide to an interim concentration of 14 mg/mL PDC-E2 peptide. The peptide dissolved in ammonium hydroxide is dialyzed against ultra-pure water using a dialysis system (SpectraFlo Dialyzer, Repligen). The pH of the dialyzed peptide solution is maintained between pH 6.0 and pH 7.0. The adjusted solution is then further diluted to the final 8.25 mg/mL PDC-E2 peptide concentration by adding water for injection (WFI) and mixed until homogenous.

### Example 2. Process for Manufacturing Tolerizing Nanoparticles Encapsulating PBC Associated Antigens (TIMP-PBC)

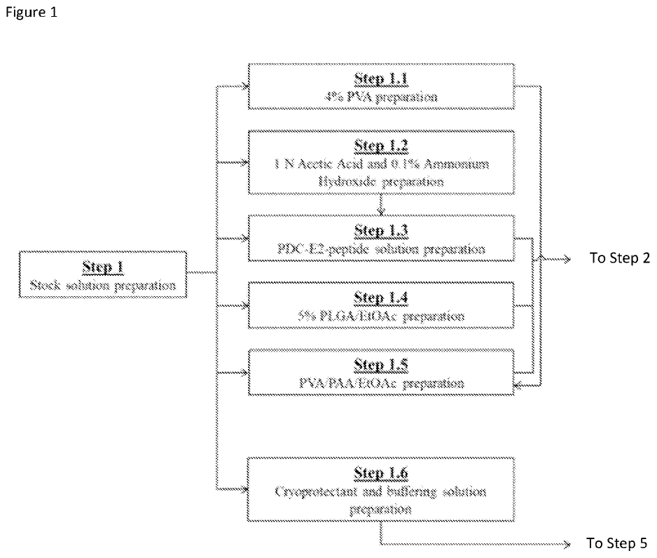

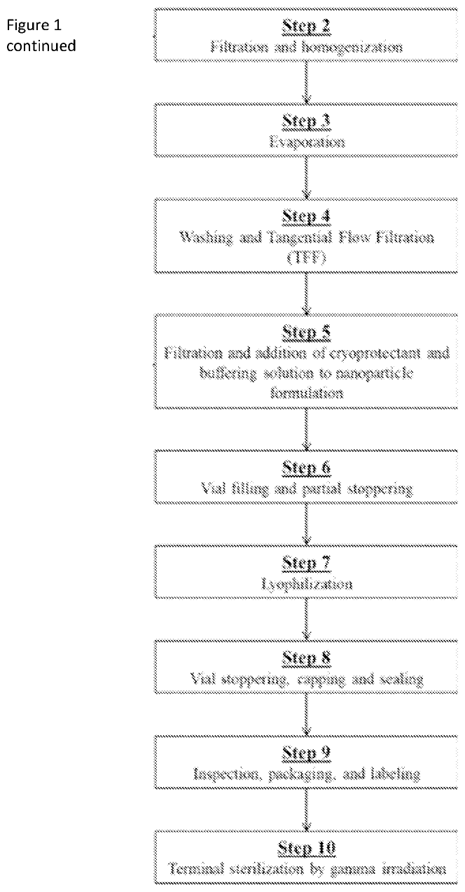

TIMP-PBC (CNP-104) was manufactured using a double-emulsion solvent evaporation process. An exemplary manufacturing process flow diagram is shown in FIG. 1. Briefly, PDC-E2 peptide was dissolved in 0.1% ammonium hydroxide and the pH is adjusted with 1N acetic acid (8.2 mg/mL) and rapidly mixed with a 5% PLGA solution (50:50; molecular weight between 10,000 to 60,000 Da) in ethyl acetate to generate a primary water-in-oil emulsion. A stock mixture of water, PVA, PAA, and ethyl acetate was generated by admixing PVA (4% in water), PAA (Sigma Aldrich, 100 kDa, 35% wt in water), and ethyl acetate to generate a blend. The composition of the PVA/PAA/ethyl acetate/water blend was maintained at a pH below 4.0. The primary emulsion was then rapidly mixed with the PVA/PAA/ethyl acetate blend to form an oil-in-water secondary emulsion. Mixing of the primary and secondary emulsions was performed by homogenization.

Solvent was removed from the secondary emulsion by evaporation under pressure for a total of at least 3-4 hours. Hardened nanoparticles were then washed in sterile water and concentrated by filtration using a 20 μm filter. Cryoprotectants sucrose and mannitol and buffering agent sodium citrate dihydrate were added to the hardened nanoparticles. The formulation was then lyophilized.

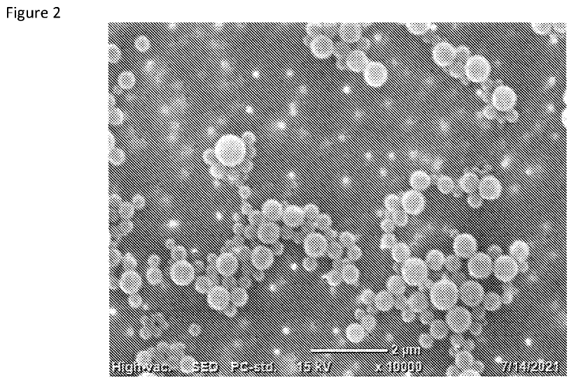

The final TIMP-PBC formulation was characterized to determine physiochemical properties such as particle diameter, zeta potential, total peptide content. The results of TIMP-PBC characterization are provided in Table 1. TIMP-PBC particles were examined by Scanning Electron Microscopy showing a homogenous composition of intact particles with smooth surfaces (FIG. 2).

TABLE 1
Physiochemical characterization of CNP-104
particles encapsulating PDC-E2 peptides.

| Nanoparticle Physiochemical Property | Result |
|---|---|
| Particle Size by Dynamic Light Scattering | 520 nm |
| Zeta Potential by Dynamic Light Scattering | −54 mV |
| Particle Size by Laser Diffraction D[4/3] | 542 nm |
| Size distribution by single particle | 520/560/630 |
| optical sensing (SPOS) | |
| D 10/D 50/D 90 | |
| PDC-E2 peptide Content by RP-HPLC | 0.5 μg/mg PLGA - 10 μg/mg PLGA |
| Particle concentration by Flow cytometry | 1.1 × 10⁹ particles/mg |
| of CNP-104 formulation (count/mg) | |
| Total particle count per 100 mg vial | 1.1 × 10¹¹ particles |
| PDC-E2 peptide encapsulated/particle* | 0.5 femtograms/particle- |
| | 10 femtograms/particle |
| Endotoxin | <0.048 EU/mg |

*Amount of PDC-E2 peptide encapsulated in particle calculated by dividing PDC-E2 peptide content (μg/mg) by particle concentration (count/mg)

### Example 3. Pharmaceutical Compositions of TIMP-PBC

Pharmaceutical formulations or compositions of TIMP-PBC comprise individual components listed in Table 2.

TABLE 2

| Component | 80 g batch | 160 g batch |
|---|---|---|
| PLGA nanoparticles | 30-40% | 30-40% |
| Sucrose | 30-40% | 30-40% |
| Mannitol | 20-30% | 20-30% |
| Sodium citrate | 1-10% | 1-10% |
| PBC associated | 0.5 μg/mg to 10 | 0.5 μg/mg to 10 |
| antigens | μg/mg of PLGA | μg/mg of PLGA |

It is understood that every embodiment of the disclosure described herein may optionally be combined with any one or more of the other embodiments described herein. Every patent literature and every non-patent literature cited herein are incorporated herein by reference in their entirety.

It is understood, therefore, that this invention is not limited to the particular embodiments disclosed, but is intended to cover all modifications which are within the spirit and scope of the invention as defined by the appended claims; the above description, and/or shown in the attached drawings. Consequently, only such limitations as appear in the appended claims should be placed on the disclosure.

### REFERENCES

1. Lu M, Zhou Y, Haller I V, et al. Increasing Prevalence of Primary Biliary Cholangitis and Reduced Mortality With Treatment. Clin Gastroenterol Hepatol. 2018; 16(8):1342-1350.e1341.
2. Webb G J, Siminovitch K A, Hirschfield G M. The immunogenetics of primary biliary cirrhosis: A comprehensive review. J Autoimmun. 2015; 64:42-52.
3. Mejía SP, Sánchez A, Vasquez V, Orozco J. Functional Nanocarriers for Delivering Itraconazole Against Fungal Intracellular Infections. Front Pharmacol. 2021 Jun. 28; 12:685391.
4. Zielińska A, Carreiró F, Oliveira A M, Neves A, Pires B, Venkatesh D N, Durazzo A, Lucarini M, Eder P, Silva A M, Santini A, Souto E B. Polymeric Nanoparticles: Production, Characterization, Toxicology and Ecotoxicology. Molecules. 2020 Aug. 15; 25(16):3731.

What is claimed:

1. A method for preparing a composition comprising particles encapsulating one or more primary biliary cholangitis (PBC)-associated antigens, the method comprising:
a. generating an aqueous solution of one or more PBC associated antigens;
b. generating a primary emulsion by mixing an aqueous solution of step (a) with a polymer resulting in a primary emulsion;
c. mixing the primary emulsion with a mixture comprising one or more surfactants and/or stabilizers to form a secondary emulsion; and
d. hardening the secondary emulsion by evaporation resulting in hardened polymeric nanoparticles encapsulating one or more PBC associated antigens within their cores.

2. The method of claim 1, further comprising (e) filtering, washing, and concentrating the nanoparticles.

3. The method of claim 1 or 2 further comprising (f) freeze drying the nanoparticles to form a composition.

4. The method of any one of claims 1-3, wherein the aqueous solution of step (a) comprises a solvent.

5. The method of any one of claims 1-4, wherein the solvent of step (a) is selected from the group consisting of acetaldehyde, acetone, acetonitrile, acetic acid, 1,2-butanediol, 1,3-butanediol, 1,4-butanedoil, 2-butoxyethanol, diethanolamine, diethylenetriamine, dimethoxyethane, dimethylformamide, 1,1-dimethylformamide, 1,1-dimethylhydrazine, 1,2-dimethylhydrazine, dimethyl sulfoxide, 1,4-dioxane, formic acid, ethanol, ethylamine, ethylene glycol, furfuryl alcohol, glycerol, isopropanol, methanol, methyl diethanolamine, methyl isocyanide, N-methyl-2-pyrrolidone, 1-propanol, 1,3-propanediol, 1,5-pentanediol, 2-propanol, propylene glycol, pyridine, tetrahydrofuran, triethylene glycol, benzoic acid, ascorbic acid, trichloroacetic acid, dichloroacetic acid, chloroacetic acid, trifluoroacetic acid, fluoroacetic acid, tartaric acid, lactic acid, gluconic acid, methanesulfonic acid, ethanesulfonic acid, benzenesulfonic acid, p-toluenesulfonic acid, trifluoromethanesulfonic acid, polystyrene sulfonic acid, oxalic acid, carbonic acid, methylamine, diethylamine, pyridine, sulfuric acid, hydrochloric acid, nitric acid, hydrobromic, hydroiodic acid, hypochlorous acid, chloric acid, chloric acid, perchloric acid, fluorosulfuric acid, fluoroantimonic acid, fluoroboric acid, hexafluorophosphoric acid, chromic acid, phosphoric acid, hydrofluoric acid, boric acid, barium hydroxide, calcium hydroxide, chromium hydroxide, potassium hydroxide, ammonium hydroxide, zinc hydroxide, sodium bicarbonate, sodium hydroxide, magnesium hydroxide, ammonium bicarbonate, ammonia, aluminium hydroxide, sodium carbonate, magnesium hydroxide, zinc hydroxide, ferrous hydroxide, lithium hydroxide, and rubidium hydroxide.

6. The method of any one of claims 1-5, wherein the solvent of step (a) has a pH between 1.0 and 14.0.

7. The method of any one of claims 1-6, wherein the initial concentration of the one or more PBC associated antigens dissolved in step (a) is between 0.1 to 100 mg/mL.

8. The method of any one of claims 1-7, wherein the pH of the solution of step (a) is adjusted to between 1.0 and 7.0.

9. The method of any one of claims 1-8, wherein the final concentration of one or more PBC associated antigens in step (a) is between 0.1 to 100 mg/mL.

10. The method of any one of claims 1-9, wherein the mixtures of step (b) and step (c) include one or more solvents.

11. The method of claim 10, wherein the one or more solvents is an organic solvent or an inorganic solvent.

12. The method of any one of claims 10-11, wherein the one or more solvents of step (b) and step (c) are the same.

13. The method of any one of claims 10-11, wherein the one or more solvents of step (b) and step (c) are different.

14. The method of any one of claims 11-13, wherein the organic solvent is selected from the group consisting of acetic acid, acetone, ethanol, methylene chloride, dimethyl sulfoxide (DMSO), ethyl acetate, dimethylformamide, tetrahydrofuran, and chloroform.

15. The method of any one of claims 1-14, wherein the emulsion resulting from step (b) is a water-in-oil emulsion.

16. The method of any one of claims 1-15, wherein the emulsion resulting from step (c) is an oil-in-water emulsion.

17. The method of any one of claims 1-16, wherein the polymer of step (b) is a biodegradable polymer.

18. The method of claim 15, wherein the biodegradable polymer is selected from the group consisting of polyglycolic acid (PGA), polylactic acid (PLA), polysebacic acid (PSA), poly(lactic-co-glycolic) (PLGA), poly(lactic-co-sebacic) acid (PLSA), poly(glycolic-co-sebacic) acid (PGSA), polypropylene sulfide, poly (caprolactone), chitosan, a polysaccharide, and a lipid.

19. The method of any one of claims 1-18, wherein the surfactant or stabilizer of step (c) is anionic, cationic, zwitterionic, or nonionic.

20. The method of claim 19, wherein the surfactant and/or stabilizer is selected from the group consisting of a poloxamer, a polyamine, PEG, Tween-80, gelatin, dextran, pluronic L-63, pluronic F-68, pluronic 188, pluronic F-127, PVA, PAA, methylcellulose, lecithin, DMAB, PEMA, vitamin E TPGS (D-a-tocopheryl polyethylene glycol 1000 succinate), hyaluronic acid, poly amino acids, poly lysine, poly arginine, poly aspartic acid, poly glutamic acid, polyserine, polythreonine, polytyrosine, poly cysteine, enantiomers of poly amino acids, sodium cholate, methylcellulose, hydroxyethylcellulose, hydroxyprolylcellulose, hydroxypropylmethylcellulose, gelatin, a carbomer, and a sulfate polymer.

21. The method of any one of claims 1-20, wherein the primary emulsion of step (b) is obtained by homogenization.

22. The method of any one of claims 1-20, wherein the primary emulsion of step (b) is obtained by sonication.

23. The method of any one of claims 1-22, wherein the secondary emulsion of step (c) is obtained by homogenization.

24. The method of any one of claims 1-22, wherein the secondary emulsion of step (c) is obtained by sonication.

25. The method of any one of claims 1-24, wherein the pH of the emulsion of step (b) is less than pH 4.0.

26. The method of claim 21 or 23, wherein homogenization is performed for 5, 10, 15, 20, 25, 30, 30, 40, 45, 60, 90, 120, 150, 180, 210, 240, 270, 300, 330, 360, 390, 420, 450, 480, 510, 540, 570, or 600 seconds.

27. The method of claim 22 or 24, wherein sonication is performed for 5, 10, 15, 20, 25, 30, 30, 40, 45, 60, 90, 120, 150, 180, 210, 240, 270, 300, 330, 360, 390, 420, 450, 480, 510, 540, 570, or 600 seconds.

28. The method of any one of claims 1-27, wherein the hardening of nanoparticles in step (d) is performed by evaporation of the solvent.

29. The method of claim 28, wherein evaporation is active evaporation or passive evaporation.

30. The method of claim 29, wherein active evaporation is vacuum-driven evaporation.

31. The method of claim 30, wherein the vacuum-driven evaporation is performed under high pressure or low pressure.

32. The method of claim 29, wherein passive evaporation is performed by stirring.

33. The method of any one of claims 28-32, wherein evaporation is performed for 0.25, 0.5, 1, 2, 3, 4, 5, 6, 7, 8, 9, 10, 11, 12, 13, 14, 15, 16, 17, 18, 19, 20, 21, 22, 23, 24, 48, 72, or 96 hours.

34. The method of any one of claims 2-33, wherein filtering, washing, and concentrating the nanoparticles in step (c) is performed by filtration, gel filtration, membrane filtration, dialysis, centrifugation, chromatography, density gradient centrifugation, or combinations thereof.

35. The method of any one of claims 1-34, wherein the one or more PBC associated antigen content encapsulated within the particle composition is about 0.1 to 100 μg/mg.

36. The method of any one of claims 1-35, wherein the one or more PBC associated antigens are selected from the group consisting pyruvate dehydrogenase complex E2 subunit (PDC-E2), gp210, nucleoporin 62, Sp100, PML, CENP A, CENP B, CENP C, dsDNA, histone, branched-chain 2-oxo-acid dehydrogenase complex (BCOADC), lipoamide acyltransferase component of branched-chain alpha-keto acid dehydrogenase complex (BCOADC-E2), dihydrolipoyllysine-residue succinyltransferase component of 2-oxoglutarate dehydrogenase complex (OGDC-E2), sulfite oxidase, outer mitochondrial membrane, glycogen phosphorylase, sarcosine dehydrogenase, smooth muscle protein, soluble liver antigen, liver/kidney microsome, centromere protein, tubulin, actin, vimentin, desmin, cytokeratin, F-actin, UDP glucuronosyltransferase Family 1 Member A Complex (UGTA1), formiminotransferase cyclodeaminase, asialoglycoprotein receptor (ASGPR), cardiolipin, h-Lamp-2, proteinase 3, CYP 2C9, CYP 2A6, and CYP P450 2D6, 2-octynamide, 2-nonynamide, Escherichia coli PDC-E2, Novosphingobium aromaticivorans proteins Novo 1, Novo 2, Novo 3, Novo 4, or Lactobacillus delbrueckii beta-galactosidase, 2-octynamide, 2-nonynamide, Escherichia coli PDC-E2, Escherichia coli ATP dependent ClpX, Escherichia coli periplasmic maltose-binding protein, Escherichia coli ATP dependent helicase Hrp, Escherichia coli fatty acid oxidation complex alpha, Escherichia coli ppGpp synthetase II, Escherichia coli nitrate reductase 2, Helicobacter pylori urease beta subunit, Pseudomonas aeruginosa diaminopimelate decarboxylase, human cytomegalovirus capsid assembly protein UL47, Haemophilus influenzae 1-RNA (uracil-5-)-methyltransferase.

37. The method of claim 36, wherein the PBC associated antigen comprises PDC-E2₁₅₅₋₁₈₅.

38. The method of claim 36, wherein the PBC associated antigen is PDC-E2₁₅₅₋₁₈₅ set out in SEQ ID NO: 1.

39. A method for manufacturing a composition comprising negatively charged particles encapsulating pyruvate dehydrogenase complex E2 subunit (PDC-E2)₁₅₅₋₁₈₅ peptides comprising:
(a) generating an aqueous solution of PDC-E2₁₅₅₋₁₈₅ peptide between pH 1.0 and pH 7.0
(b) generating a primary emulsion by homogenizing the aqueous solution of PDC-E2₁₅₅₋₁₈₅ peptides of step (a) with an oil phase including PLGA dissolved in ethyl acetate;
(c) homogenizing the primary emulsion of step (b) with a mixture comprising PAA, PVA and ethyl acetate to form a secondary emulsion; and
(d) hardening the secondary emulsion of step (c) by evaporation resulting in hardened polymeric nanoparticles encapsulating PDC-E2₁₅₅₋₁₈₅ peptides within their cores.

40. The method of claim 39 further comprising (e) filtering, washing, and concentrating the nanoparticles.

41. The method of claim 40 further comprising (f) addition of sodium citrate, mannitol and sucrose to the nanoparticles and freeze drying the nanoparticles.

42. The method of any one of claims 39-41, wherein the acetic acid is between about 0.5N to about 2N.

43. The method of any one of claims 39-42, wherein the PLGA is a 5% PLGA solution comprising 50:50 PLGA having a molecular weight between 10,000 to 60,000 Da.

44. The method of any one of claims 1-43, wherein the particles have a negative zeta potential.

45. The method of claim 44, wherein the zeta potential of the particles is between about 0 and −100 mV.

46. The method of claim 44, wherein the zeta potential of the particles is between about −30 and −80 mV.

47. The method of any one of claims 1-46, wherein the particles have a diameter of between about 0.3 μm to 3μ m.

48. The method of claim 47, wherein the particles have a diameter of between about 0.3 μm to 1 μm.

49. The method of claim 48, wherein the particles have a diameter of between about 0.4 μm to 1 μm.

50. The method of any one of claims 1-49, wherein at least 90% of the particles have a diameter of between about 0.3 μm to 3 μm.

51. The method of claim 50, wherein at least 90% of the particles have a diameter of between about 0.3 μm to 1 μm.

52. The method of claim 50, wherein at least 90% of the particles have a diameter of between about 0.4 μm to 1 μm.

53. The method of any one of claims 1-52, wherein at least 50% of the particles have a diameter of between about 0.3 μm to 3 μm.

54. The method of claim 53, wherein at least 50% of the particles have a diameter of between about 0.3 μm to 1 μm.

55. The method of claim 54, wherein at least 50% of the particles have a diameter of between about 0.4 μm to 1 μm.

56. The method of any one of claims 1-55, wherein at least 10% of the particles have a diameter of between about 0.3 μm to 3 μm.

57. The method of claim 56, wherein at least 10% of the particles have a diameter of between about 0.3 μm to 1 μm.

58. The method of any one of the preceding claims, wherein the manufacturing batch size is between 0.01 g to 100 kg.

59. The method of claim 58, wherein the manufacturing batch size is 0.01 g, 0.1 g, 10 g, 20 g, 40 g, 60 g, 80 g, 100 g, 160 g, 240 g, 320 g, 400 g, 480 g, 560 g, 640 g, 720 g, 800 g, 1000 g, 5 kg, 10 kg, 50 kg, or 100 kg.

60. The method of any one of claims 1-59, wherein the aqueous solution of step (a) comprises one or more acids and/or one or more base.

61. The method of any one of claims 1-60, wherein the aqueous solution of step (a) comprises a base selected from the group consisting of ammonium hydroxide, barium hydroxide, calcium hydroxide, chromium hydroxide, potassium hydroxide, zinc hydroxide, barium hydroxide, sodium bicarbonate, methylamine, diethylamine, sodium hydroxide, magnesium hydroxide, ammonium bicarbonate, ammonia aluminium hydroxide, sodium carbonate, magnesium hydroxide, zinc hydroxide, ferrous hydroxide, acetone, lithium hydroxide, pyridine, and rubidium hydroxide.

62. The method of any one of claims 1-60, wherein the aqueous solution of step (a) comprises an acid selected from the group consisting of acetic acid, sulfuric acid, hydrochloric acid, nitric acid, formic acid, benzoic acid, ascorbic acid, trichloroacetic acid, dichloroacetic acid, chloroacetic acid, trifluoroacetic acid, fluoroacetic acid, tartaric acid, lactic acid, gluconic acid, methanesulfonic acid, ethanesulfonic acid, benzenesulfonic acid, p-toluenesulfonic acid, trifluoromethanesulfonic acid, polystyrene sulfonic acid, hydrobromic acid, hydroiodic acid, hypochlorous acid, chloric acid, chloric acid, perchloric acid, fluorosulfuric acid, fluoroantimonic acid, fluoroboric acid, hexafluorophosphoric acid, chromic acid, phosphoric acid, hydrofluoric acid, oxalic acid, boric acid, carbonic acid.

63. The method of claim 62, wherein the acid comprises acetic acid.

64. A composition comprising particles encapsulating one or more primary biliary cholangitis (PBC)-associated antigens made by the method of any one of claims 1-63.

65. The composition of claim 64, wherein the PBC associated antigen comprises pyruvate dehydrogenase complex E2 subunit (PDC-E2)₁₅₅₋₁₈₅ peptide.

66. The composition of claim 64 or 65, wherein the PBC associated antigen is pyruvate dehydrogenase complex E2 subunit (PDC-E2)₁₅₅₋₁₈₅ peptide set out in SEQ ID NO: 1.

67. The composition of any one of claims 64-66, further comprising a pharmaceutically acceptable carrier, diluent or excipient.

68. The composition of claim 67, wherein the excipients are sucrose, mannitol, and sodium citrate.

69. A pharmaceutical composition comprising negatively charged particles encapsulating pyruvate dehydrogenase complex E2 subunit (PDC-E2)₁₅₅₋₁₈₅ peptide, sucrose, mannitol, and sodium citrate.

70. The pharmaceutical composition of claim 69, wherein negatively charged particles comprise poly (lactic co-glycolic acid) (PLGA) and PDC-E2₁₅₅₋₁₈₅ peptide, wherein the PDC-E2₁₅₅₋₁₈₅ peptide amount is between 0.1 μg/mg and 100 μg/mg.

71. The pharmaceutical composition of claim 69 or 70, wherein the PDC-E2₁₅₅₋₁₈₅ peptide amount is between 1 μg/mg to 5 μg/mg.

72. The pharmaceutical composition of any one of claims 69-71, wherein the PDC-E2₁₅₅₋₁₈₅ peptide comprises amino acid sequence KVGEKLSEGDLLAEIETDKATIGFEVQEEGY (SEQ ID NO: 1).

73. The pharmaceutical composition of any one of claims 65-68, wherein the PLGA is carboxylated PLGA.

74. The pharmaceutical composition of claims 69-73, wherein the zeta potential of the particles is between about 0 and −100 mV.

75. The pharmaceutical composition of claim 74, wherein the zeta potential of the particles is between about −30 and −80 mV.

76. The pharmaceutical composition of any one of claims 69-75, wherein the particles have a diameter of between about 0.3 μm to 3 μm.

77. The pharmaceutical composition of any one of claims 69-76, wherein the sucrose concentration is between 30% w/v to 40% w/v.

78. The pharmaceutical composition of any one of claims 69-77, wherein the mannitol concentration is between 20% w/v to 30% w/v.

79. The pharmaceutical composition of any one of claims 69-78, wherein the sodium citrate concentration is between 0.5% w/v to 3.5% w/v.

80. The pharmaceutical composition of any one of claims 69-79, wherein the particles are lyophilized.

81. A method of treating a subject having primary biliary cholangitis (PBC) comprising administering a composition of any one of claims 66-68 or a pharmaceutical composition of any one of claims 69-80.

82. A composition of any one of claims 66-68 or a pharmaceutical composition of any one of claims 69-80 for treating primary biliary cholangitis (PBC) in a subject.

83. Use of a composition of any one of claims 66-68 or a pharmaceutical composition of any one of claims 69-80 in the manufacture of a medicament for treating primary biliary cholangitis (PBC) in a subject.
